SEQUENCE LISTING (6 sequences)

SEQ ID NO: 1 - DNA, 870 bp, unidentified
  source 1..870: mol_type = unassigned DNA
atggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtga

SEQ ID NO: 2 - DNA, 1653 bp, unidentified
  source 1..1653: mol_type = unassigned DNA
atggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaa

SEQ ID NO: 3 - DNA, 360 bp, unidentified
  source 1..360: mol_type = unassigned DNA
atgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagtaa

SEQ ID NO: 4 - DNA, 801 bp, unidentified
  source 1..801: mol_type = unassigned DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggagatccaaaaaagaagagaaaggtaggcgatccaaaaaagaagagaaaggtaggtgatccaaaaaagaagagaaaggtataa

SEQ ID NO: 5 - DNA, 1656 bp, unidentified
  source 1..1656: mol_type = unassigned DNA
atggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgggctccggcgagggcaggggaagtcttctaacatgcggggacgtggaggaaaatcccggcccactcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 6 - DNA, 720 bp, unidentified
  source 1..720: mol_type = unassigned DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

(19) United States
(12) Patent Application Publication
(10) Pub. No.: US 2023/0364267 A1
(43) Pub. Date: Nov. 16, 2023

(54) PRE-CLINICAL MODIFIED RNA APPROACHES USED IN LARGE ANIMALS FOR MUSCLE AND VASCULAR REGENERATION

(71) Applicant: THE UAB RESEARCH FOUNDATION, Birmingham, AL (US)

(72) Inventors: Jianyi Zhang, Vestavia, AL (US); Yang Zhou, Vestavia, AL (US); Jiacheng Sun, Birmingham, AL (US); Lu WANG, Birmingham, AL (US)

(21) Appl. No.: 18/315,677

(22) Filed: May 11, 2023

Related U.S. Application Data
(60) Provisional application No. 63/364,664, filed on May 13, 2022.

(51) Int. Cl. A61K 48/00; A61P 9/10

(52) U.S. Cl. CPC A61K 48/0066; A61K 48/0075; A61P 9/10

(57) ABSTRACT

In one aspect, the disclosure relates to a synthetic modified mRNA (modRNA) and a novel gene therapy approach using the same that offers efficient, transient, safe, nonimmunogenic, and controlled mRNA delivery to the heart tissue without any risk of genomic integration. In a further aspect, the disclosure relates to methods of using the modRNA to achieve transient and exclusive overexpression of CCND2 only in cardiomyocytes, increasing cell cycle markers, enhancing cardiac function, and promoting myocardial repair. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/364,664, filed on May 13, 2022, which is incorporated herein by reference in its entirety.

### CROSS-REFERENCE TO SEQUENCE LISTING

This application contains a sequence listing filed in ST.26 format entitled “222119-1160_Sequence_Listing.xml” created on May 5, 2023, and having a file size of 12 kB. The content of the sequence listing is incorporated herein in its entirety.

### BACKGROUND

Patients with severe acute myocardial infarction (AMI) often progress to end-stage congestive heart failure (CHF), which is one of the most significant problems in public health. From the molecular and cellular perspective, heart failure occurs due to the loss of the contractile unit of the left ventricle: cardiomyocytes (CMs). Mammalian CMs exit the cell cycle shortly after birth, but the results from previous studies with neonatal pig indicated that when AMI was induced on postnatal day 1 (P1), CMs re-entered the cell cycle and proliferated, leading to the complete restoration of cardiac function with a little evidence of scarring by P30. Some anecdotal evidence in pediatric patient suggests that the regenerative capacity of newborn infant hearts is similar. However, mammalian CM exit the cell cycle within a few days after birth, so less than 1% of the CM in adult human hearts are replaced each year. Studies in adult mice suggest that cardiomyocyte proliferation increases only marginally in response to cardiac injury. This meager proliferative capacity cannot repair the damage caused by AMI in adult mammals.

Investigations in mouse, rat, and pig MI models have targeted many of the pathways that regulate the cell cycle of CM in an attempt to promote cardiomyocyte proliferation and improve recovery from myocardial injury. For example, Cyclin D2 (CCND2) in humans controls the G1-to-S phase transition in CM, and targeted CM cyclin D2 expression has been associated with improvements in infarct size and cardiac performance. Furthermore, when human induced-pluripotent stem cells (hiPSCs) were engineered to MHC driven overexpress CCND2 CM (CCND2-OEhiPSC-CMs), and the CCND2-OEhiPSC-CMs were transplanted into infarcted mouse hearts, the small number of hiPSC-CMs that were engrafted at the site of administration proliferated and repopulated the myocardial scar, thereby reducing infarct size and improving cardiac performance. However, the methods used to manipulate the expression of regulatory molecules are often accompanied by safety concerns that impede their translation to clinical use. Viral-based therapies, especially those that can become integrated into the genome of the host cell (e.g., adeno-associated virus [AAV]), may promote excessive and enduring expression of the gene of interest, which could result in cardiac hypertrophy and induce uncontrolled cardiomyocyte proliferation, increasing the risk of arrhythmia. Although nonintegrating lentiviral vectors (NILV) are expressed only transiently in vivo, their off the target side-effects, and therefore, the safety in humans remains largely unknown.

Despite advances in cardiac regeneration and repair research, there is still a scarcity of gene delivery systems and methods that are both potent and efficacious at achieving selective repair of cardiac tissue without overexpressing proteins in non-target tissue. These needs and other needs are satisfied by the present disclosure.

### SUMMARY

In accordance with the purpose(s) of the present disclosure, as embodied and broadly described herein, the disclosure, in one aspect, relates to a synthetic modified mRNA (modRNA) and a novel gene therapy approach using the same that offers efficient, transient, safe, nonimmunogenic, and controlled mRNA delivery to the heart tissue without any risk of genomic integration. In a further aspect, the disclosure relates to methods of using the modRNA to achieve transient and exclusive overexpression of CCND2 only in cardiomyocytes, increasing cell cycle markers, enhancing cardiac function, and promoting myocardial repair.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1J show CCND2-CM SMRTs promoted proliferation in cultured post-mitotic CM. (FIG. 1A) The Cardiomyocyte Specific ModRNA Translation System (CM SMRTs) consists of two modRNA constructs as illustrated. (B-C) Co-cultures of post-mitotic hiPSC-CMs and (FIG. 1B) ECs or (FIG. 1C) FBs were transfected with GFP-modRNA or GFP-CM SMRTs; then, the cultured cells were immunofluorescently stained for the expression GFP and markers for CM (cardiac troponin T [cTnT] and a sarcomeric actin [aSA]), ECs (von Willebrand factor [vWF]), and/or FBs (vimentin). Nuclei were counterstained with DAPI. Scale bar=50 μm. (FIGS. 1D-1H) hiPSC-CMs were transfected with Luc-CM SMRTs or CCND2-CM SMRTs, stained for cTnT expression and for (FIG. 1D) CCND2 expression, (FIG. 1E) the expression of Ki67 (a proliferation marker), (FIG. 1F) BrdU incorporation (a marker for cell-cycle S-phase), (FIG. 1G) PH3 (a marker for cell-cycle G2/M phase transition), or (FIG. 1H) the expression of AuB (a cytokinesis marker). Nuclei were counterstained with DAPI, and the percentage of hiPSC-CMs that were positive for CCND2, Ki67, BrdU, or PH3, and for G2/Prophase-, Anaphase-, or Abscission-like AuB expression was determined via immunofluorescence staining. *P<0.05, ***P<0.001; Scale bar=50 μm. (FIG. 1I) Time-lapse images were obtained of hiPSC-CMs that underwent cell division (yellow arrowheads) after treatment with the CCND2-CM SMRTs. (FIG. 1J) hiPSC-CM cell counts were quantified for 7 days after treatment with the Luc-CM or CCND2-CM SMRTs. Scale bar=50 μm; n=4; *P<0.05, **P<0.01.

FIGS. 2A-2I show intramyocardial injections of CCND2-CM SMRTs promoted cardiomyocyte proliferation and improved measures of cardiac function, infarct size, and hypertrophy in a mouse MI model. (FIG. 2A) Mice with induced MI received intramyocardial injections of GFP-CM SMRTs (100 μg GFP-modRNA and 50 μg L7Ae-modRNA) and were sacrificed three days later; then, sections of heart tissue from the site of administration were stained for cTnT and GFP expression, and nuclei were counterstained with DAPI. Scale bar=50 μm. (FIG. 2B) The experimental protocol in the mouse MI model is shown as a schematic. (FIGS. 2C-2E) Sections of heart tissue from the border zone of the infarct were collected from mice one week after MI surgery and stained for the presence of cTnT and (FIG. 2C) Ki67, (FIG. 2D) PH3, or (FIG. 2E) AuB; then, cardiomyocyte proliferation, cell-cycle activity, and cytokinesis were quantified as the percentages of cTnT-positive cells that were also positive for Ki67, PH3, and symmetric and asymmetric AuB, respectively. Scale bar=50 μm for (FIG. 2C); Scale bar=10 μm for (FIG. 2D); Scale bar=10 μm for (FIG. 2E); **P<0.01, ***P<0.001. (FIG. 2F) Left-ventricular (LV) ejection fraction (EF) and (FIG. 2G) fractional shortening (FS) were calculated from echocardiographic images obtained 1 day before MI induction and 0.5, 2, 4, 7, 14, and 28 days afterward. *P<0.05 vs. Vehicle, **P<0.01 vs. Vehicle, ***P<0.001 vs. Vehicle; ^{###}P<0.001 vs. L7Ae-modRNA; ^{&&&}P<0.001 vs. Luc-CM SMRTs. (FIG. 2H) LVs collected from mice at week 4 were cut into sections and stained with Sirius Red and Fast Green to visualize fibrotic (red) and normal (green) tissue; then, infarct size was quantified as the ratio of the length of LV scar arc to the circumference of the LV and presented as a percentage. Scale bar=1 mm; *P<0.05, **P<0.01, ***P<0.001.

FIGS. 3A-3D show intramyocardial injections of CCND2-CM SMRTs promoted cardiomyocyte cell-cycle activity and proliferation in a pig AMI model. (FIG. 3A) The protocol for experiments in the pig MI model is shown as a schematic. (FIGS. 3B-3D) Sections of heart tissue from the border zone and remote zone of the infarct were collected from pigs three days after MI and stained for the presence of cTnT and (FIG. 3B) Ki67, (FIG. 3C) PH3, or (FIG. 3D) AuB; then, cardiomyocyte proliferation, cell-cycle activity, and cytokinesis were quantified as the percentages of cTnT-positive cells that were also positive for Ki67, PH3, and symmetric and asymmetric AuB, respectively. Scale bar=50 μm for B-C; Scale bar=10 μm for D; n=3 animals per group; *P<0.05, **P<0.01, ***P<0.001.

FIGS. 4A-4M show intramyocardial injections of CCND2-CM SMRTs improved global and regional measures of cardiac function in infarcted pig hearts. (FIGS. 4A-4C) Echocardiographic images (FIG. 4A) were obtained for pigs in the CCND2-CM SMRTs, nGFP-CM SMRTs, and Vehicle groups on Day 10 and Day 28 after AMI induction and used to calculate (FIG. 4B) LVEF and (FIG. 4C) LVFS. **P<0.01, ***P<0.001. (FIGS. 4D-4H) cMRI was performed on Day 28 and used to calculate (FIG. 4E) LVEF, (FIG. 4F) Stroke Volume, (FIG. 4G) End-systolic Volume, and (FIG. 4H) End-diastolic Volume. *P<0.05, **P<0.01, ***P<0.001. (FIGS. 4I-4M) LGE-cMRI was performed on Day 28 and used to calculate radial strain (ε_{RR}) and LV wall thickening (LVWT). (FIG. 4I) Corresponding T1 (left) and LGE (right) images are displayed for a pig in the Vehicle group; the infarcted region and the border and remote zones are identified with arrows in the LGE image. Six-segment curves corresponding to (FIG. 4J) LV radial strain (ε_{RR}) and (FIG. 4K) LV wall thickening (LVWT) were plotted, and (FIGS. 4L-4M) the area under the curve (AUC) of both parameters were calculated. IS: inferoseptal, AS: anteroseptal, A: anterior, AL: anterolateral, IL: inferolateral, I: inferior. n=5 for Vehicle group; n=6 for nGFP-CM SMRTs group; n=7 for CCND2-CM SMRTs group. *P<0.05, **P<0.01, ***P<0.001 vs. Vehicle group; ^{#}P<0.05, ^{##}P<0.01, ^{###}P<0.001 vs. nGFP-CM SMRTs group.

FIGS. 5A-5J show intramyocardial injections of CCND2-CM SMRTs reduced infarct size after MI induction in pigs. (FIG. 5A) LGE-cMRI images were obtained in pigs on Day 28 after MI induction; the endocardium is marked with a red line, the epicardium is marked with a blue line, and the infarct area is shaded in yellow. (FIGS. 5B-5D) LGE-cMRI images were used to calculate (FIG. 5B) infarct size, (FIG. 5C) infarct mass, and (FIG. 5D) infarct mass percentage relative to the LV weight; size and mass measurements were presented as a percentage of the entire LV. **P<0.01, ***P<0.001. (FIG. 5E) An LGE-cMRI image is displayed showing the region of the scar core (white zone; shaded in yellow) and the surrounding region of necrotic and viable tissue (gray zone; shaded in purple). (FIGS. 5F-5H) The mass of the (FIG. 5F) infarct core and (FIG. 5G) gray zone, and (FIG. 5H) the ratio of the masses of the core and gray zones were calculated from LGE-cMRI images. **P<0.01, ***P<0.001. (FIG. 5I) LVs were collected on Day 28 and cut into five circular sections from the apex to the base. Scale bar=2 cm. (FIG. 5J) Sections of the infarct core and the surrounding border zone from infarcted cardiac rings 2, 3, 4 were stained with Picro-Sirius Red and Fast Green to visualize fibrotic (red) and normal (green) tissue; then infarct size was quantified as the ratio of the area of the fibrotic region to total area of the tissue and presented as a percentage. Scale bar=1 cm; ***P<0.001. n=5 for Vehicle group; n=6 for nGFP-CM SMRTs group; n=7 for CCND2-CM SMRTs group.

FIGS. 6A-6D show intramyocardial injections of CCND2-CM SMRTs promoted cardiomyocyte proliferation for less than 28 days and did not increase the risk of arrhythmia in infarcted pig hearts. (FIGS. 6A-6B) Sections from border zone and remote zone were stained with cTnT and wheat germ agglutinin (WGA) to visualize cardiomyocytes and borders. Nuclei were counterstained with DAPI. (FIG. 6A) Cardiomyocyte cross-sectional size and (FIG. 6B) the percentage of CMs exhibiting mono-, bi-, and multi-nucleation were quantified. Scale bar=50 μm; *P<0.05, **P<0.01, ***P<0.001. (FIGS. 6B-6C) Sections from the border and remote zones of pig hearts were collected on Day 28 and stained for the presence of cTnT and (FIG. 6B) Ki67 or (FIG. 6C) PH3; then, cardiomyocyte proliferation and cell-cycle activity were quantified as the percentages of cTnT-positive cells that were also positive for Ki67 and PH3, respectively. Scale bar=50 μm. (FIG. 6D) Programmed electrical stimulation (PES) was performed in pig hearts before sacrifice on Day 28. Hearts were paced at 400 ms with additional stimuli provided at progressively shorter intervals; PES was halted immediately after an episode of ventricular arrhythmia (VA) was induced. n=5 for Vehicle group; n=6 for nGFP-CM SMRTs group; n=7 for CCND2-CM SMRTs group.

FIG. 7 shows an integrity analysis of modRNAs. The integrity of the modRNA constructs was validated with a bioanalyzer for L7Ae-modRNA, CCND2-modRNA, GFP-modRNA, nGFP-modRNA, Luc-modRNA, and CCND2-T2A-nGFP-modRNA.

FIGS. 8A-8C show GFP expression in cultured hiPSC-CMs began to decline 3 days after treatment with GFP-modRNA. (FIG. 8A) hiPSC-CMs were transfected with the indicated concentrations of GFP modRNA; 24 hours later, the proportion of hiPSC-CMs that were positive for GFP expression was determined via flow cytometry. (FIG. 8B) The proportion of hiPSC-CMs that were positive for GFP expression was determined via flow cytometry at daily intervals for up to one week after transfection with the optimized dose (3 μg) of GFP modRNA. (FIG. 8C) Forty-eight hours after transfection with GFP modRNA or Vehicle (Lipofectamine MessengerMax), hiPSC-CMs were stained for the presence of GFP and cTnT, and apoptotic hiPSC-CMs were identified via TUNEL-staining; then, apoptosis was quantified as the percentage of cells that were positive for TUNEL. Scale bar=50 μm.

FIGS. 9A-9B show CCND2 CM-SMRTs treatment efficiently and transiently promotes CCND2 expression in cultured post-mitotic hiPSC-CMs. (FIG. 9A) CCND2 protein abundance was evaluated at the indicated time points via Western blot in post-mitotic (60-day-old) hiPSC-CMs that were genetically modified to overexpress CCND2 (CCND2-OEhiPSC-CMs) or had been treated with the CCND2 SMRTs on Day 0; GAPDH abundance was also evaluated to control for unequal loading. (FIG. 9B) The results from Western blot analyses were quantified via densitometry analysis and normalized to GAPDH levels; n=3 samples per group. ***P<0.001.

FIGS. 10A-10B show Intramyocardial injections of CCND2-CM SMRTs reduced the mortality of mice after MI and promoted cardiac CCND2 expression. (FIG. 10A) The survival of mice in all four groups that underwent MI induction surgery is displayed in a Kaplan-Meier plot. (FIG. 10B) CCND2 expression was evaluated via Western blot for mice in the CCND2-CM SMRTs, Vehicle, and Sham groups on Day 3 after MI or Sham surgery. Glyceraldehyde phosphate dehydrogenase (GAPDH) abundance was also evaluated to control for unequal loading, and then the results were quantified via densitometry analysis and normalized to GAPDH levels. n=2 animals per group. *P<0.05, **P<0.01.

FIGS. 11A-11B show Intramyocardial injections of the CCND2-CM SMRTs after MI transiently increased the expression of proliferation markers in CMs. (FIGS. 11A-11B) Sections of heart tissue from the border zone of the infarct were collected from mice 7, 14, and 28 days after MI and stained for the presence of cTnT and (FIG. 11A) Ki67, or (FIG. 11B) PH3; then, cardiomyocyte proliferation and cell-cycle activity were quantified as the percentages of cTnT-positive cells that were also positive for Ki67 and PH3. ***P<0.001.

FIGS. 12A-12C show Intramyocardial injections of CCND2-CM SMRTs promoted LV systolic function and reduced cardiac hypertrophy after MI in mice. (FIG. 12A) Representative echocardiographic images collected 1 day before, 0.5, 2, 4, 7, 14, and 28 days after MI surgery and treatment. (FIG. 12B) Left ventricle (LV) end-systolic volume (LVESV) and end-diastolic volume (LVEDV) were measured by echography at the aforementioned timepoints and normalized to the body weight (BW) of each mouse. *P<0.05 vs. Vehicle, **P<0.01 vs. Vehicle, ***P<0.001 vs. Vehicle; #P<0.05 vs. L7Ae-modRNA, ##P<0.01 vs. L7Ae-modRNA; &P<0.05 vs. Luc-CM SMRTs. (FIG. 12C) Hearts were explanted from mice sacrificed on Day 28; then, cardiac hypertrophy was evaluated by determining the heart-weight to bodyweight (HW/BW) ratio for each group. The number of animals per group is indicated. ***P<0.001.

FIG. 13 shows Intramyocardial injections of the CCND2-CM SMRTs reduced infarct size in a mouse MI model. LVs collected from mice at week 4 were cut into sections and stained with Sirius Red and Fast Green to visualize fibrotic (red) and normal (green) tissue. Images are displayed for all sections used to quantify infarct size in FIG. 2H.

FIGS. 14A-14B show CCND2 and nGFP expression was co-localized in the CMs of infarcted pig hearts after CCND2-nGFP-CM SMRTs administration. (FIG. 14A) Sections of heart tissue from the site of CCND2-nGFP-CM SMRTs administration in infarcted pig hearts were stained for cTnT, GFP, and CCND2 expression, and nuclei were counterstained with DAPI; Scale bar=500 μm. (FIG. 14B) The boxed regions in panel A are displayed at higher magnification; Scale bar=50 μm.

FIGS. 15A-15B show Intramyocardial injections of CCND2-CM SMRTs after MI promoted CCND2 expression in the hearts of pigs but not in any other organ. (FIG. 15A) CCND2 protein abundance was evaluated via Western blot in the organs of pigs sacrificed on Day 3 after MI induction and treatment; GAPDH abundance was also evaluated to control for unequal loading. (FIG. 15B) The results from Western blot analyses were quantified via densitometry analysis and normalized to GAPDH levels; n=3 animals per group. ***P<0.001.

FIG. 16 shows Indices of LV radial strain and LV wall thickening were generally greater in pigs treated with CCND2-CM SMRTs after MI induction than in the Luc-CM SMRTs— and Vehicle-treatment groups. LV Radial strain (εRR) and LV wall thickening (LVWT) were calculated for each segment in the American Heart Association's 16-segment model of the LV; results are displayed as bullseye graphs.

FIGS. 17A-17B show Intramyocardial injections of CCND2-CM SMRTs after MI reduced cardiac hypertrophy. (FIGS. 17A-17B) Heart weight (HW), left ventricular weight (LV W) and body weight (BW) of each animal were measured to calculate (FIG. 17A) heart weight/body weight ratio and (FIG. 17B) left ventricular weight/body weight ratio. *P<0.05, **P<0.01.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

modRNA technology provides an alternative strategy for transiently inducing gene expression that is efficient, titratable, and minimally immunogenic with bell-shaped pharmacokinetics and no risk of genomic integration. Furthermore, the safety profile of modRNA was thoroughly studied during the development of mRNA-based vaccines against SARS-CoV-2. Disclosed herein is a Cardiomyocyte Specific Modified mRNA Translation System (CM SMRTs) that allows gene expression exclusively in cardiomyocytes (CM). In one aspect, the disclosed CM SMRTs have been used to drive CCND2 transient expression in endogenous CM in both mouse and pig acute myocardial infarction (AMI) models and has been shown to activate the cell cycle of CMs and improve the recovery of left ventricular (LV) injury in hearts with AMI.

In one aspect, disclosed herein is a synthetic modified mRNA (modRNA) system for delivery of at least one cell cycle regulator gene to cardiac cells, wherein the modRNA system includes a composition including at least a first modRNA and a second modRNA. Further in this aspect, the first modRNA includes an mRNA sequence complementary to SEQ ID NO. 3 and recognition sequences for the microRNAs miR-1 and miR-208. In an aspect, miR-1 and miR-208 are cardiomyocyte (CM)-specific microRNAs and the combination of miR-1 and miR-208 is not found in non-cardiac cells. In one aspect, the recognition sequences for miR-1 and miR-208 can be located anywhere in the first modRNA but, in some aspects, they are located 3′ to the mRNA sequence complementary to SEQ ID NO. 3. In an aspect, SEQ ID NO. 3 is a DNA sequence encoding the archaeal large ribosomal subunit. Thus, in this aspect, targeting CM-specific microRNAs allows the disclosed systems and methods to be active in target cardiac cells and not in other cell types. In another aspect, the second modRNA includes a kink-turn motif and an mRNA sequence complementary to SEQ ID NO. 1. In an aspect, the kink-turn motif is a common structural motif in RNA that introduces a tight kink into the helical axis. In a further aspect, kink-turns play an important role in RNA structures and can serve as binding sites for a number of proteins, including, but not limited to, L7Ae. In an aspect, any kink-turn motif capable of serving as a binding or recognition site for L7Ae is contemplated in the systems and methods described herein and should be considered disclosed. In another aspect, the kink-turn motif can be located anywhere in the second modRNA but, in some aspects, is located 5′ to the mRNA sequence complementary to SEQ ID NO. 1. In one aspect, SEQ ID NO. 1 is a DNA sequence encoding cell cycle regulator CCND2. In any of these aspects, the disclosed modRNA system is inactive in non-cardiac cells.

In any of these aspects, the first and/or second modRNA can include N1-methylpseudouridine residues in place of one or more uridine residues, or in place of all uridine residues. Further in this aspect, N1-methylpseudouridine as a component of an exogenously-administered RNA stimulates less of an innate immune response to the RNA than use of uridine residues at the same location. In another aspect, N1-methylpseudouridine can be incorporated into the modRNAs disclosed herein by any method known in the art including, but not limited to, incorporation of N1-methylpseudouridine triphosphate in a mixture of nucleotides used for standard molecular biological methods of RNA synthesis. In an aspect, the first and second modRNAs disclosed herein are nonimmunogenic.

In one aspect, the cardiac cells can be cardiac fibroblasts, cardiomyocytes, endothelial cells, other cardiac cells, or any combination thereof. In an aspect, the composition can include about 1.5 μg of the first modRNA and about 3 μg of the second modRNA.

Also disclosed herein is a method for improving at least one measure of cardiac health in a subject, the method including administering the disclosed modRNA system to the subject. In some aspects, the method can be performed after myocardial infarction (MI) or another source of cardiac damage. In an aspect, in the disclosed method, the modRNA system can be administered to the subject via intromyocardial injection. In any of these aspects, neither the first modRNA nor the second modRNA is genomically integrated into the subject.

In one aspect, performing the method induces transient overexpression of cell cycle regulator CCND2 in at least one cardiac cell type such as, for example, cardiac fibroblasts, cardiomyocytes, endothelial cells, other cardiac cells, or any combination thereof. In an aspect, the transient expression may peak at about 2 days after performing the method. Further in this aspect, the transient overexpression declines to substantially background levels about 14 days after performing the method.

In one aspect, the at least one measure of cardiac health includes increased expression of cell cycle markers, enhancement of cardiac function, promotion of myocardial repair, or any combination thereof. In another aspect, the subject can be a mammal such as, for example, a human, mouse, or pig.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a cardiomyocyte,” “a cyclin D protein,” or “an mRNA,” include, but are not limited to, mixtures or combinations of two or more such cardiomyocytes, cyclin D proteins, or mRNAs, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

All experiments and procedures involving animals were approved by the Institutional Animal Care and Use Committee (Animal Protocol Number 20216) of the University of Alabama at Birmingham, School of Medicine and were consistent with the Guidelines for the Care and Use of Laboratory Animals published by the US National Institutes of Health (2011). The raw data supporting the conclusions of this article will be made available by the authors, without undue reservation.

modRNA Synthesis and Transfection

modRNAs were synthesized from plasmid templates containing open reading frame sequences of the gene of interest (Table 1) via in vitro transcription (IVT) as described previously. IVT was performed with a customized ribonucleotide blend of CleanCap Reagent AG, m7G(5′)ppp(5′)(2′OMeA)pG (TriLink Biotechnologies), guanosine triphosphate (GTP; Invitrogen), adenosine triphosphate (ATP; Invitrogen), cytidine triphosphate (CTP; Invitrogen), N1-methylpseudouridine-5′-triphosphate (TriLink Biotechnologies), T7 enzyme, a tailed DNA template containing the T7 promoter, and a buffer system. mRNA was purified with a Megaclear kit (Life Technology), characterized using Agilent 2100 bioanalyzer at Heflin Center for Genomic Science at the University of Alabama at Birmingham, and concentrated with Amicon Ultra-2 30 k Centrifugal Filters (UFC203024, Millipore). In vitro transfection of modRNA was performed with Lipofectamine MessengerMax Reagent (Invitrogen, LMRNA015) by the manufacturer's instructions, and a biocompatible sucrose-citrate buffer was used for in vivo transfection.

TABLE 1
Open Reading Frame Sequences Used for modRNA Production

| | | SEQ |
|---|---|---|
| | | ID |
| Gene | Open Reading Frame | NO. |
| CCND2 | atggagctgctgtgccacgaggtggacccggtccgcagggccgtgcgggaccgc | 1 |
| | aacctgctccgagacgaccgcgtcctgcagaacctgctcaccatcgaggagcgct | |
| | accttccgcagtgctcctacttcaagtgcgtgcagaaggacatccaaccctacatgc | |
| | gcagaatggtggccacctggatgctggaggtctgtgaggaacagaagtgcgaag | |
| | aagaggtcttccctctggccatgaattacctggaccgtttcttggctggggtcccgact | |
| | ccgaagtcccatctgcaactcctgggtgctgtctgcatgttcctggcctccaaactca | |
| | aagagaccagcccgctgaccgcggagaagctgtgcatttacaccgacaactcca | |
| | tcaagcctcaggagctgctggagtgggaactggtggtgctggggaagttgaagtg | |
| | gaacctggcagctgtcactcctcatgacttcattgagcacatcttgcgcaagctgccc | |
| | cagcagcgggagaagctgtctctgatccgcaagcatgctcagaccttcattgctctg | |
| | tgtgccaccgactttaagtttgccatgtacccaccgtcgatgatcgcaactggaagtg | |
| | tgggagcagccatctgtgggctccagcaggatgaggaagtgagctcgctcacttgt | |
| | gatgccctgactgagctgctggctaagatcaccaacacagacgtggattgtctcaa | |
| | agcttgccaggagcagattgaggcggtgctcctcaatagcctgcagcagtaccgtc | |
| | aggaccaacgtgacggatccaagtcggaggatgaactggaccaagccagcacc | |
| | cctacagacgtgcgggatatcgacctgtga | |
| Luc | atggccgatgctaagaacattaagaagggccctgctcccttctaccctctggaggat | 2 |
| | ggcaccgctggcgagcagctgcacaaggccatgaagaggtatgccctggtgcct | |
| | ggcaccattgccttcaccgatgcccacattgaggtggacatcacctatgccgagta | |
| | cttcgagatgtctgtgcgcctggccgaggccatgaagaggtacggcctgaacacc | |
| | aaccaccgcatcgtggtgtgctctgagaactctctgcagttcttcatgccagtgctgg | |
| | gcgccctgttcatcggagtggccgtggcccctgctaacgacatttacaacgagcgc | |
| | gagctgctgaacagcatgggcatttctcagcctaccgtggtgttcgtgtctaagaag | |
| | ggcctgcagaagatcctgaacgtgcagaagaagctgcctatcatccagaagatc | |
| | atcatcatggactctaagaccgactaccagggcttccagagcatgtacacattcgtg | |
| | acatctcatctgcctcctggcttcaacgagtacgacttcgtgccagagtctttcgaca | |
| | gggacaaaaccattgccctgatcatgaacagctctgggtctaccggcctgcctaag | |
| | ggcgtggccctgcctcatcgcaccgcctgtgtgcgcttctctcacgcccgcgaccct | |
| | attttcggcaaccagatcatccccgacaccgctattctgagcgtggtgccattccacc | |
| | acggcttcggcatgttcaccaccctgggctacctgatttgcggctttcgggtggtgctg | |
| | atgtaccgcttcgaggaggagctgttcctgcgcagcctgcaagactacaaaattca | |
| | gtctgccctgctggtgccaaccctgttcagcttcttcgctaagagcaccctgatcgac | |
| | aagtacgacctgtctaacctgcacgagattgcctctggcggcgccccactgtctaa | |
| | ggaggtgggcgaagccgtggccaagcgctttcatctgccaggcatccgccaggg | |
| | ctacggcctgaccgagacaaccagcgccattctgattaccccagagggcgacga | |
| | caagcctggcgccgtgggcaaggtggtgccattcttcgaggccaaggtggtggac | |
| | ctggacaccggcaagaccctgggagtgaaccagcgcggcgagctgtgtgtgcgc | |
| | ggccctatgattatgtccggctacgtgaataaccctgaggccacaaacgccctgat | |
| | cgacaaggacggctggctgcactctggcgacattgcctactgggacgaggacga | |
| | gcacttcttcatcgtggaccgcctgaagtctctgatcaagtacaagggctaccaggt | |
| | ggccccagccgagctggagtctatcctgctgcagcaccctaacattttcgacgccg | |
| | gagtggccggcctgcccgacgacgatgccggcgagctgcctgccgccgtcgtcgt | |
| | gctggaacacggcaagaccatgaccgagaaggagatcgtggactatgtggcca | |
| | gccaggtgacaaccgccaagaagctgcgcggcggagtggtgttcgtggacgag | |
| | gtgcccaagggcctgaccggcaagctggacgcccgcaagatccgcgagatcct | |
| | gatcaaggctaagaaaggcggcaagatcgccgtgtaa | |
| L7Ae | atgtacgtgagatttgaggttcctgaggacatgcagaacgaagctctgagtctgctg | 3 |
| | gagaaggttagggagagcggtaaggtaaagaaaggtaccaacgagacgacaa | |
| | aggctgtggagaggggactggcaaagctcgtttacatcgcagaggatgttgaccc | |
| | gcctgagatcgttgctcatctgcccctcctctgcgaggagaagaatgtgccgtacatt | |
| | tacgttaaaagcaagaacgaccttggaagggctgtgggcattgaggtgccatgcg | |
| | cttcggcagcgataatcaacgagggagagctgagaaaggagcttggaagccttgt | |
| | ggagaagattaaaggccttcagaagtaa | |
| nGFP | atggtgagcaagggcgaggagctgttcaccggggtggtgcccatcctggtcgagc | 4 |
| | tggacggcgacgtaaacggccacaagttcagcgtgtccggcgagggcgagggc | |
| | gatgccacctacggcaagctgaccctgaagttcatctgcaccaccggcaagctgc | |
| | ccgtgccctggcccaccctcgtgaccaccctgacctacggcgtgcagtgcttcagc | |
| | cgctaccccgaccacatgaagcagcacgacttcttcaagtccgccatgcccgaag | |
| | gctacgtccaggagcgcaccatcttcttcaaggacgacggcaactacaagaccc | |
| | gcgccgaggtgaagttcgagggcgacaccctggtgaaccgcatcgagctgaag | |
| | ggcatcgacttcaaggaggacggcaacatcctggggcacaagctggagtacaa | |
| | ctacaacagccacaacgtctatatcatggccgacaagcagaagaacggcatcaa | |
| | ggtgaacttcaagatccgccacaacatcgaggacggcagcgtgcagctcgccga | |
| | ccactaccagcagaacacccccatcggcgacggccccgtgctgctgcccgacaa | |
| | ccactacctgagcacccagtccgccctgagcaaagaccccaacgagaagcgcg | |
| | atcacatggtcctgctggagttcgtgaccgccgccgggatcactctcggcatggac | |
| | gagctgtacaagggagatccaaaaaagaagagaaaggtaggcgatccaaaaa | |
| | agaagagaaaggtaggtgatccaaaaaagaagagaaaggtataa | |
| nGFP- | atggagctgctgtgccacgaggtggacccggtccgcagggccgtgcgggaccgc | 5 |
| CCND2 | aacctgctccgagacgaccgcgtcctgcagaacctgctcaccatcgaggagcgct | |
| | accttccgcagtgctcctacttcaagtgcgtgcagaaggacatccaaccctacatgc | |
| | gcagaatggtggccacctggatgctggaggtctgtgaggaacagaagtgcgaag | |
| | aagaggtcttccctctggccatgaattacctggaccgtttcttggctggggtcccgact | |
| | ccgaagtcccatctgcaactcctgggtgctgtctgcatgttcctggcctccaaactca | |
| | aagagaccagcccgctgaccgcggagaagctgtgcatttacaccgacaactcca | |
| | tcaagcctcaggagctgctggagtgggaactggtggtgctggggaagttgaagtg | |
| | gaacctggcagctgtcactcctcatgacttcattgagcacatcttgcgcaagctgccc | |
| | cagcagcgggagaagctgtctctgatccgcaagcatgctcagaccttcattgctctg | |
| | tgtgccaccgactttaagtttgccatgtacccaccgtcgatgatcgcaactggaagtg | |
| | tgggagcagccatctgtgggctccagcaggatgaggaagtgagctcgctcacttgt | |
| | gatgccctgactgagctgctggctaagatcaccaacacagacgtggattgtctcaa | |
| | agcttgccaggagcagattgaggcggtgctcctcaatagcctgcagcagtaccgtc | |
| | aggaccaacgtgacggatccaagtcggaggatgaactggaccaagccagcacc | |
| | cctacagacgtgcgggatatcgacctgggctccggcgagggcaggggaagtcttc | |
| | taacatgcggggacgtggaggaaaatcccggcccactcgagatggtgagcaag | |
| | ggcgaggagctgttcaccggggtggtgcccatcctggtcgagctggacggcgacg | |
| | taaacggccacaagttcagcgtgtccggcgagggcgagggcgatgccacctacg | |
| | gcaagctgaccctgaagttcatctgcaccaccggcaagctgcccgtgccctggcc | |
| | caccctcgtgaccaccctgacctacggcgtgcagtgcttcagccgctaccccgacc | |
| | acatgaagcagcacgacttcttcaagtccgccatgcccgaaggctacgtccagga | |
| | gcgcaccatcttcttcaaggacgacggcaactacaagacccgcgccgaggtgaa | |
| | gttcgagggcgacaccctggtgaaccgcatcgagctgaagggcatcgacttcaag | |
| | gaggacggcaacatcctggggcacaagctggagtacaactacaacagccacaa | |
| | cgtctatatcatggccgacaagcagaagaacggcatcaaggtgaacttcaagatc | |
| | cgccacaacatcgaggacggcagcgtgcagctcgccgaccactaccagcagaa | |
| | cacccccatcggcgacggccccgtgctgctgcccgacaaccactacctgagcac | |
| | ccagtccgccctgagcaaagaccccaacgagaagcgcgatcacatggtcctgct | |
| | ggagttcgtgaccgccgccgggatcactctcggcatggacgagctgtacaagtaa | |
| GFP | atggtgagcaagggcgaggagctgttcaccggggtggtgcccatcctggtcgagc | 6 |
| | tggacggcgacgtaaacggccacaagttcagcgtgtccggcgagggcgagggc | |
| | gatgccacctacggcaagctgaccctgaagttcatctgcaccaccggcaagctgc | |
| | ccgtgccctggcccaccctcgtgaccaccctgacctacggcgtgcagtgcttcagc | |
| | cgctaccccgaccacatgaagcagcacgacttcttcaagtccgccatgcccgaag | |
| | gctacgtccaggagcgcaccatcttcttcaaggacgacggcaactacaagaccc | |
| | gcgccgaggtgaagttcgagggcgacaccctggtgaaccgcatcgagctgaag | |
| | ggcatcgacttcaaggaggacggcaacatcctggggcacaagctggagtacaa | |
| | ctacaacagccacaacgtctatatcatggccgacaagcagaagaacggcatcaa | |
| | ggtgaacttcaagatccgccacaacatcgaggacggcagcgtgcagctcgccga | |
| | ccactaccagcagaacacccccatcggcgacggccccgtgctgctgcccgacaa | |
| | ccactacctgagcacccagtccgccctgagcaaagaccccaacgagaagcgcg | |
| | atcacatggtcctgctggagttcgtgaccgccgccgggatcactctcggcatggac | |
| | gagctgtacaagtaa | |

### Cardiomyocyte Culture and Proliferation Measurement

Human cardiac fibroblasts induced pluripotent stem cells (hiPSCs) were maintained in mTeSR Plus medium (STEMCELL Technologies). The α-myosin heavy chain (α-MHC) promoter—driven CCND2 CMs (CCND2-OEhiPSC-CMs) were derived as previously described in details using GiWi protocol for cardiomyocyte differentiation. Both wildtype hiPSC-CMs and CCND2-OEhiPSC-CMs were cultured for 60 days after differentiation, which time point has minimal mitotic activity.

To calculate the CMs cell proliferation, 2×10⁴ hiPSC-CMs were seeded in 12-well plate and treated with CCND2-CM SMRTs or Luc-CM SMRTs. Cell numbers from day 0 to day 7 were quantified by an automatic cell counter (Countess 3, Invitrogen). Time lapse movies showing CCND2-CM SMRTs—treated hiPSC-CMs dividing were taken by Lionheart FX automated microscope (Agilent, Santa Clara, CA, USA) for 48 hours.

### Flow Cytometry

Flow cytometry was performed as described previously to detect GFP-positive hiPSC-CM ratio in dose-dependent study and time-course study. To determine the optimized dosage for modRNA transfection, different dosages of GFP-modRNA were transfected to 5×10⁴ hiPSC-CMs seeded in 24-well plate 24 hours prior to flow cytometry analysis. Time-course study was performed by transfecting 3 μg GFP-modRNA to 5×10⁴ hiPSC-CMs seeded in 24-well plate. Then, hiPSC-CMs were trypsinized into individual cells, fixed in fixation and permeabilization solution (51-2090KZ, BD Biosciences) for 30 minutes at 4° C., blocked in Human BD Fc Block (564219, BD Biosciences) at room temperature for 10 minutes, incubated with fluorescent conjugated antibodies (Table 2) or isotype control antibodies at room temperature for 40 minutes, resuspended in wash buffer (554723, BD Biosciences), and evaluated with an LSR Fortessa instrument (BD Biosciences, USA).

TABLE 2
Antibodies

| | | | | |
|---|---|---|---|---|
| Reagent | Manufacturer | Catalog No. | Type | Dilution |

Primary Antibodies

| | | | | |
|---|---|---|---|---|
| Human Troponin T | R&D Systems | MAB1874 | Mouse monoclonal | 1:50 |
| (Cardiac) Antibody | | | | |
| Recombinant Anti- | Abcam | ab91605 | Rabbit monoclonal | 1:200 |
| Cardiac Troponin T | | | | |
| antibody | | | | |
| Monoclonal Anti-α- | Sigma-Aldrich | A7811 | Mouse monoclonal | 1:200 |
| Actinin (Sarcomeric) | | | | |
| antibody | | | | |
| Anti-GFP antibody | Abcam | ab5450 | Goat monoclonal | 1:500 |
| Anti-GFP antibody | Abcam | ab6673 | Goat monoclonal | 1:200 |
| Anti-phospho-Histone | Milipore Sigma | 06-570 | Rabbit monoclonal | 1:2000 |
| H3 (Ser10) Antibody, | | | | |
| Mitosis Marker | | | | |
| Aura B/AIM1 | Cell Signaling | #3094 | Mouse monoclonal | 1:50 |
| Antibody | Technology | | | |
| Anti- AIM-1 (Aurora | BD Transduction | 611082 | Mouse monoclonal | 1:200 |
| B) | Laboratories | | | |
| Anti-Ki-67 Antibody, | Milipore Sigma | MAB4190 | Mouse monoclonal | 1:300 |
| clone Ki-S5 | | | | |
| Anti-Ki67 antibody | Abcam | ab16667 | Rabbit monoclonal | 1:200 |
| [SP6] | | | | |
| Cyclin D2 | Cell Signaling | D52F9 | Rabbit monoclonal | 1:200 |
| | Technology | | | |
| GFP Polyclonal | Thermofisher | A-21311 | Rabbit monoclonal | 1:200 |
| Antibody, Alexa | Scientific | | | |
| Fluor ™ 488 | | | | |
| Anti-Von Willebrand | Abcam | ab6994 | Rabbit monoclonal | 1:200 |
| Factor antibody | | | | |
| Recombinant Anti- | Abcam | ab92547 | Rabbit monoclonal | 1:200 |
| Vimentin antibody | | | | |
| Alexa Fluor 488 | Thermofisher | W11261 | | 1:200 |
| conjugated wheat | Scientific | | | |
| germ agglutinin | | | | |
| (WGA) | | | | |
| BrdU | Sigma-Aldrich | 11296736001 | Mouse monoclonal | 1:10 |

Secondary Antibodies

| | | | | |
|---|---|---|---|---|
| Cy3 AffiniPure | Jackson | 711-165-152 | Donkey | 1:200 |
| Donkey Anti-Rabbit | ImmunoResearch | | monoclonal | |
| IgG | | | | |
| Cy5 AffiniPure | Jackson | 715-175-150 | Donkey | 1:200 |
| Donkey Anti-Mouse | ImmunoResearch | | monoclonal | |
| IgG | | | | |
| Fluorescein (FITC) | Jackson | 715-095-150 | Donkey | 1:200 |
| AffiniPure Donkey | ImmunoResearch | | monoclonal | |
| Anti-Mouse IgG | | | | |
| Fluorescein (FITC) | Jackson | 705-095-003 | Donkey | 1:200 |
| AffiniPure Donkey | ImmunoResearch | | monoclonal | |
| Anti-Goat IgG | | | | |
| Alexa Fluor 488 | Jackson | 705-545-003 | Donkey | 1:200 |
| AffiniPure Donkey | ImmunoResearch | | monoclonal | |
| Anti-Goat IgG | | | | |

### Histological and Immunofluorescence Analysis

After the mouse or pig hearts were explanted, they were briefly washed by natural saline and weighted. Then, the pig cardiac tissue (infarct zone and remote zone) and mouse hearts were dehydrated by 30% sucrose buffer at 4° C. overnight, embedded in O.C.T. compound, sectioned into 10 μm slides using a cryostat (Leica), and processed for histology or immunofluorescence. Cells were seeded in chamber slides, treated with modRNA or CM SMRTs, then processed for immunofluorescent staining.

For immunofluorescent staining, cells or frozen sections were tripled washed by PBS, fixed in 4% paraformaldehyde (PFA) for 10 minutes at room temperature, permeabilized with 90% acetone for 3 minutes at −20° C., blocked with 10% donkey serum for 20 minutes, incubated with primary antibodies at 4° C. overnight, then incubated with fluorescently labeled secondary antibodies for 30 minutes at room temperature in dark. The primary and secondary antibodies are listed in Table 2. Lastly, slides are mounted with Antifade Mounting Medium containing 4,6-diamidino-2-phenylindole (DAPI; Vector Laboratories), and imaged with a confocal microscope (Olympus, Japan).

BrdU incorporation analysis was performed using 5-Bromo-2′-deoxy-uridine Labeling and Detection Kit I (Roche). Briefly, 10 μM BrdU labeling solution was added to culture medium 12 hours prior to fixation and permeabilization. Next, cells were incubated with Anti-BrdU working solution for 30 minutes at 37° C. before subjected to immunofluorescent staining as described above.

Cell apoptosis was evaluated with an In-situ Cell Death Detection Kit (12156792910; Roche Applied Science, Germany) as described previously. Briefly, after fixation and permeabilization, cells were stained with terminal deoxynucleotidyl transferase dUTP nick end labeling (TUNEL) solution (Roche) at 37° C. for 1 hour, then subjected to the following primary and secondary staining to identify cTnT. Apoptosis was quantified as the ratio of the number of TUNEL positive nuclei to the total number of nuclei.

To measure CM cross-sectional area and CM nucleation, tissue sections were incubated with WGA conjugated to Alexa Fluor® 488 (Molecular Probes, Invitrogen) diluted in PBST at room temperature for 15 minutes before they were subjected to primary/secondary antibody staining as described above.

Immunofluorescence staining was quantified in every tenth serial section from the region of interest for each heart; mouse data were collected for 25-30 short axis sections out of total 250-300 sections per entire heart, and pig data were collected from at least 60 sections from two blocks each of 300 sections in the border zone containing each side of the anterior-septal LV scar perfused by left anterior descending coronary artery (i.e. border zone); and 30 sections from the region perfused by the left circumflex coronary artery (i.e., the remote zone). Five randomly selected high-resolution (40× magnification) images were evaluated for each section, and the results were quantified with Image J software.

The infarct size was evaluated by Picro-Sirius Red/Fast Green staining as previously described56. Tissue sections were fixed in Bouin's solution and stained with Fast Green dye to identify functional myocardial tissue and with Picrosirius Red to identify scar tissue. Sections were photographed with an Olympus light microscope and analyzed with Image J software. In mouse study, infarct size was calculated in every 100th serial section from apex to base (usually 8-10 sections per heart). In pig study, fibrotic area was calculated from ring 2, ring 3 and ring 4.

Mouse MI Model and modRNA Injection

MI was induced in female and male C57BL/6J mice (stock #000664; The Jackson Laboratory) as previously described. Briefly, 8-12-week-old mice were anesthetized with inhaled 2% isoflurane, intubated, and ventilated with a small animal respirator. A left thoracotomy was performed to expose the heart, and the left anterior descending (LAD) coronary artery was permanently ligated with an 8-0 non-absorbable suture; mice in the Sham group underwent all surgical procedures for MI induction except for the ligation step. Treatments (GFP-CM SMRTs, CCND2-CM SMRTs, Luc-CM SMRTs, L7Ae modRNA, or Vehicle [sucrose-citrate buffer]) were administered to three sites around the infarcted zone immediately after LAD ligation. Mice were administered buprenorphine (0.1 mg/kg every 12 hours for 3 consecutive days) and carprofen (5 mg/kg every 12 hours for 1 day) after surgery for pain control during recovery.

Pig MI Model and modRNA Injection

MI was experimentally induced in female and male Yorkshire swine (˜15 kg, Snyder Farms, Birmingham) as previously described. Briefly, pigs were anesthetized with inhaled 2% isoflurane, intubated, and ventilated with a respirator to maintain anesthesia. A center thoracotomy was performed, and the roots of the first and second diagonal coronary arteries from the LAD coronary artery were ligated with 4.0 polypropylene sutures for 60 minutes before reperfusion. Treatments (CCND2-nGFP-CM SMRTs, CCND2-CM SMRTs, nGFP-CM SMRTs, or the delivery Vehicle) were injected immediately after reperfusion into five sites in the border zone of the injured myocardium. After surgery, the chest was closed in layers, and the animals were allowed to recover. Animals received subcutaneous injections of buprenorphine SR (0.24 mg/kg; Buprenex, Rupkitt Benckiser Pharmaceuticals Inc) every 12 hours for up to 3 days and intramuscular injections of carprofen (4 mg/kg; Rimadyl, Zoetis) every 24 hours for up to 2 days after surgery.

### Western Blot

Cell sample or tissue collected from mice or pigs were lysed with T-PER™ Tissue Protein Extraction Reagent (Thermo Scientific) containing protease-inhibitor and phosphatase-inhibitor cocktails (Sigma-Aldrich); then, protein extracts were quantified with a Pierce™ BCA Protein Assay Kit (Thermo Scientific), run on Mini-PROTEANN TGX Stain-Free Gels (Bio-Rad), and transferred to polyvinylidene difluoride (PVDF) membranes using semi-dry transfer method with Trans-Blot Turbo RTA Transfer Kit (Bio-Rad). The membranes were blocked by 5% non-fat milk for 1 hour at room temperature, incubated with primary antibodies overnight at 4° C. and with secondary antibodies for 2 hours at room temperature, and then with Immobilon™ Western Chemiluminescent HRP Substrate (Millipore Sigma) before imaged using Bio-Rad ChemiDoc Imager. The protein signal was digitized and quantified with ImageJ software.

### Echocardiography

Animals were lightly anesthetized with 1-2% inhaled isoflurane, and heart rates were stable at 400-500 bpm for mice or 60-80 bpm for pigs; then, B-mode and two-dimensional M-mode images of the heart were acquired from both the long-axis and short-axis views with high-resolution micro-ultrasound systems (for mice: Vevo 2100, VisualSonics, Inc.; for pigs: GE LOGIQ V2 Ultrasound, GE Healthcare). Data were analyzed to calculate left ventricular ejection fraction and left ventricular fractional shortening.

Cardiac Magnetic Resonance Imaging (cMRI)

cMRI was performed with a 1.5-Tesla clinical scanner (GE signa horizon software 9.1) and a phased-array 4-channel surface coil with electrocardiogram (ECG) gating as previously described. Pigs were anesthetized with 2% inhaled isoflurane and positioned in a spine position within the scanner with ECG, respiratory, and cutaneous temperature monitoring. The heart was scanned along vertical and horizontal long axis views and with a set of short axis views covering the entire LV from atrioventricular valve plane to the apex. Cine imaging was performed with the following parameters: TR=3.1 ms, TE=1.6 ms, flip angle=45°, matrix size=224×128, field of view=340×265 mm², slice thickness=8 mm (with no gap between slices); 20 phases were acquired across the cardiac cycle. Infarct size was measured via late gadolinium enhancement (LGE) cMRI (0.20 mmol/kg gadopentetate dimeglumine, intravenous bolus) with the following parameters: TR=16 ms, TE=4 ms, TI=150-300 ms (TI depends on how fast the contrast washes out of the myocardium), flip angle=20°, matrix size=256×148, field of view=320×185 mm², slice thickness=8 mm (with no gap between slices).

cMRI images were analyzed using commercially available research software package (CAAS MRV 3.4, Pie Medical Imaging, Netherlands). Global LV functional parameters (end-diastolic volume [EDV] and end-systolic volume [ESV], ejection fraction [EF], stroke volume [SV]) and regional functional parameters (left ventricular regional wall thickening [LVWT] and radial strain [εRR]) were measured as previously described. LV endocardial and epicardial borders were manually contoured on all short-axis cine images at the end-diastolic and end-systolic frames to determine the EDV and ESV, respectively, as well as EF and SV. Indexes of EDV, ESV and SV were calculated by normalizing the individual values against body surface area of the pigs. To calculate regional LV function, the middle slices (area of interest), orthogonal to LV long axis, were divided into 6 circumferential segments according to the American Heart Association 17-segment model. The left-right ventricular (LRV) junction point was defined at the inferior portion of the interventricular septum. Six segments were plotted to generate the curve and subsequently calculate the area under curve (AUC).

Post-infarction fibrosis was measured in LGE-cMRI images of the LV short-axis using the same software. The quantification of LGE was performed by manually adjusting a greyscale threshold to define areas of visually identified LGE. The proportion of infarct size was calculated by dividing the total area of LGE versus total are of LV myocardium. The infarct core and the infarct gray zone (consisting necrotic and viable myocardium surrounding the infarct core) were analyzed using the full-width half-maximum method.

### Measurement of Infarct Size

Infarct size was measured by Picro-Sirius Red/Fast Green staining in mouse model, and by LGE-cMRI in pig model using the following equation:

$$\begin{matrix}Infarct Size ( \% ) = \frac{LV Scar Surface Area}{Total LV Surface Area} \times 100 \% \\ = \frac{\sum ( LV Scar Arc Length \times Section thickness )}{\sum ( LV Circumference \times Section thickness )} \times 100 \%\end{matrix}$$

In pig model of AMI, infarct was also evaluated by quantifying the gadolinium-enhanced region (Scar mass) and the entire LV weight, to derive the Infarct Mass Percentage:

$$Infarct Mass Percentage ( \% ) = \frac{LV Scar Mass}{Total LV weight} \times 100 \%$$

### Programmed Electrical Stimulation

Programmed electrical stimulation (PES) was performed as previously described. Briefly, a 6F electrode catheter was advanced through the right femoral vein and into the right ventricular apex, and the heart was paced (51) at a cycle length of 400 ms with one to three additional stimuli (S2, S3, or S4) delivered at progressively shorter intervals. S1-S2 began at 10 ms and ended at 5 ms, and the protocol was repeated for S2-S3 and S3-S4. Programmed stimulation ceased immediately after an episode of ventricular arrhythmia (VA) began. VA episodes that lasted fewer than 15 heart beats were categorized as non-sustained VA, and episodes that lasted for 15 or more heart beats were categorized as sustained VA.

### Statistical Analysis

Data are presented as mean±SEM. Significance was determined via the Student's t-test for comparisons between two groups and via one-way analysis of variance for comparisons among three or more groups. A P-value of less than 0.05 was considered statistically significant.

### Example 2: Results

### CCND2-CM SMRTs Drives Cardiomyocyte-Specific CCND2 Expression

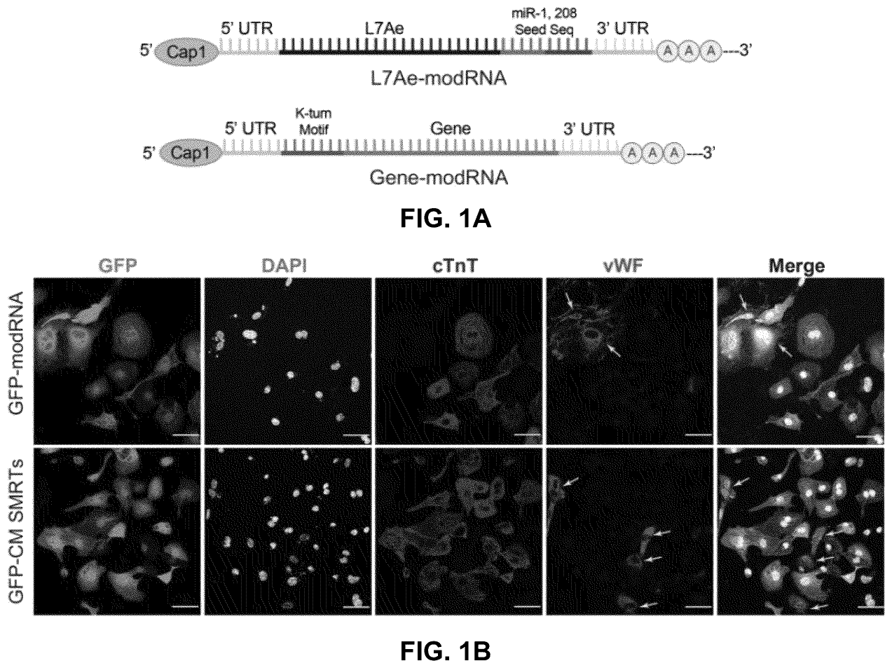

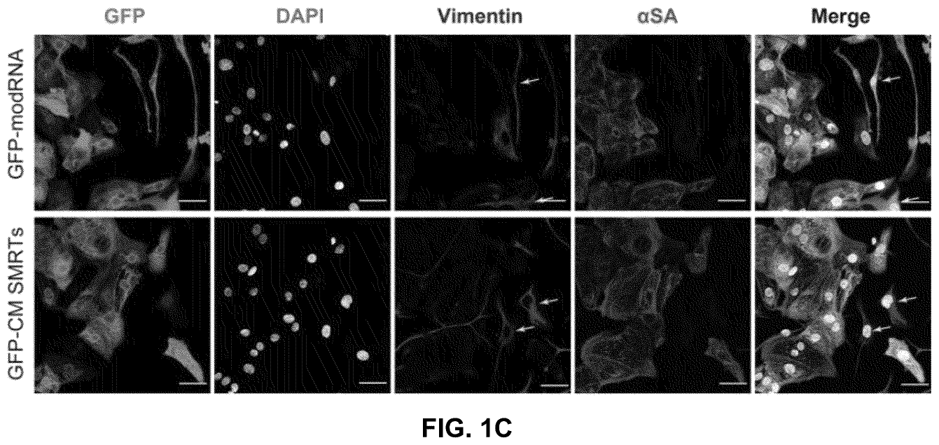

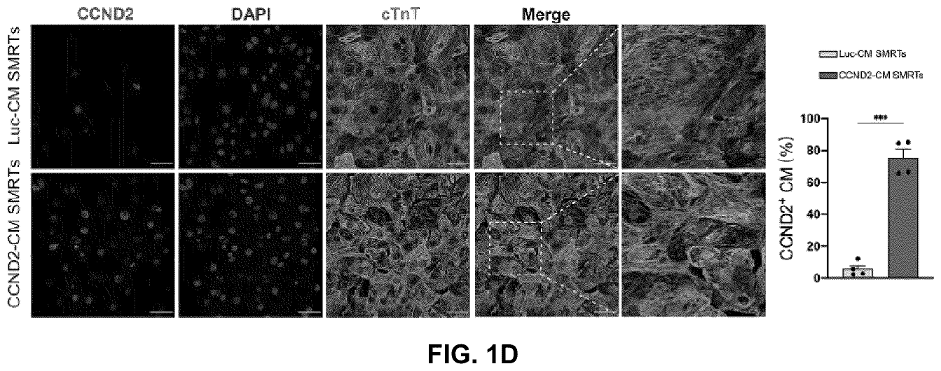

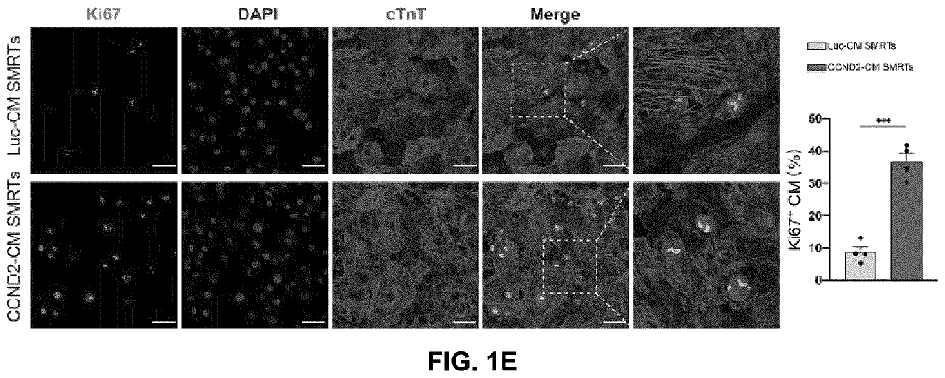

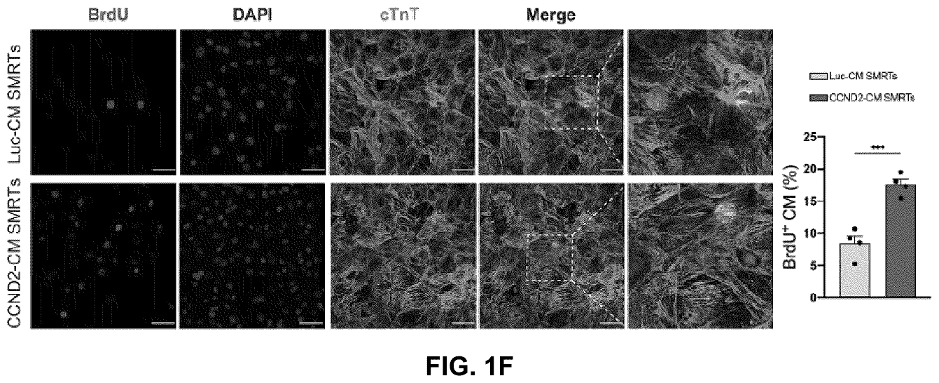

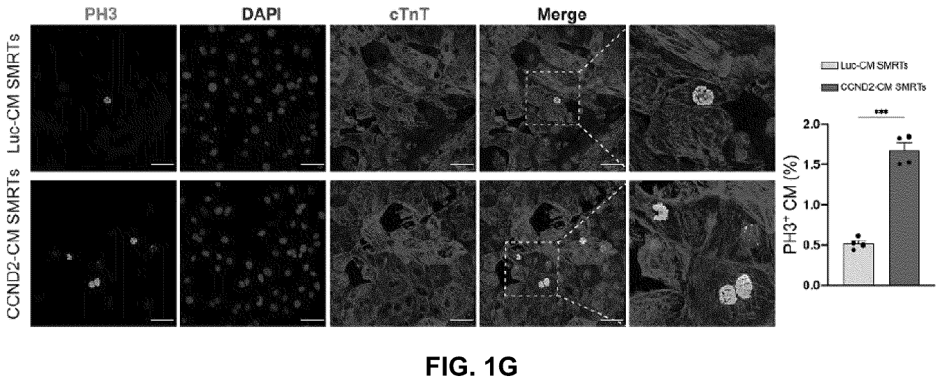

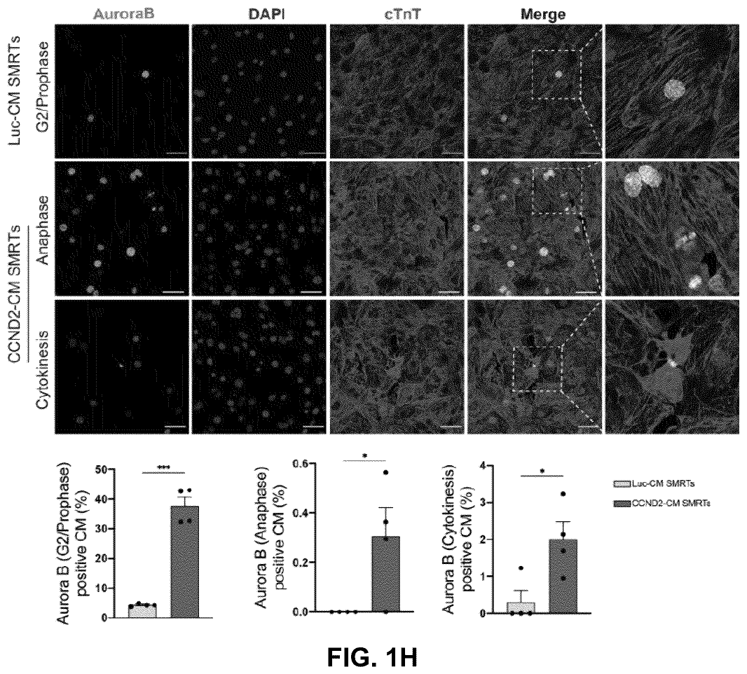

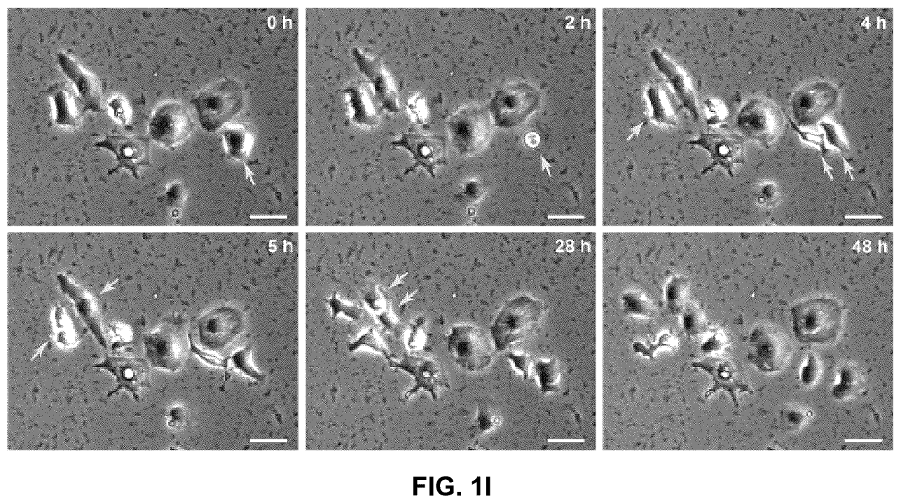

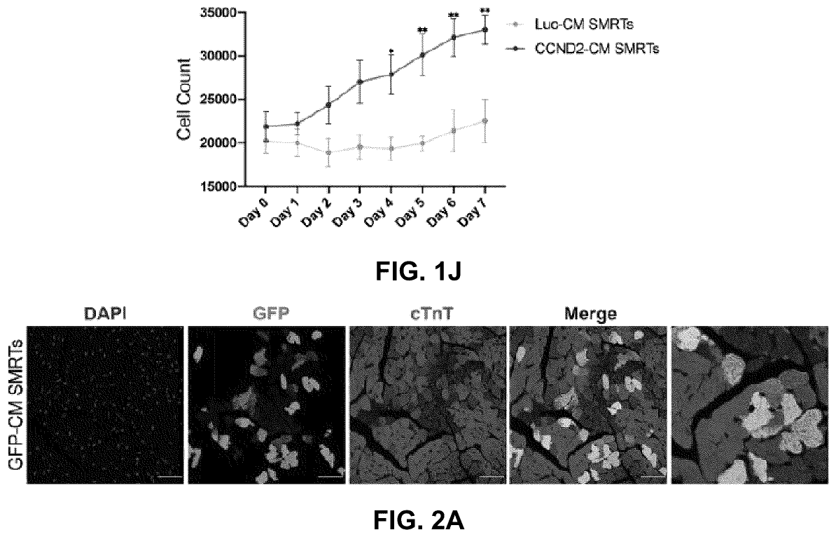

The CM SMRTs (FIG. 1A) is composed of 2 distinct modRNA constructs. One codes for the gene of interest and contains a kink-turn motif, which functions as a binding site for the archaeal ribosomal protein L7Ae, while the other codes for L7Ae and contains recognition elements for the CM-specific microRNAs miR-1 and miR-208. Thus, L7Ae suppresses translation of the gene of interest in noncardiomyocytes but is recognized and cleaved by endogenous miR-1 and -208 in cardiomyocytes, thereby facilitating cardiomyocyte-specific expression of the modRNA transcript.

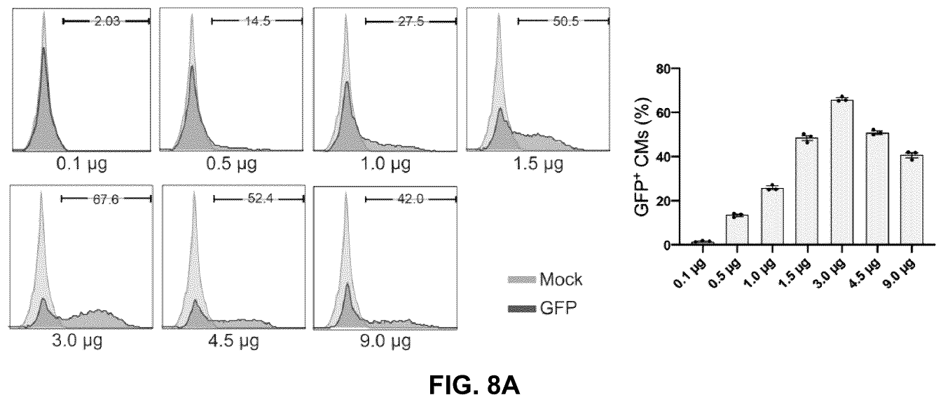

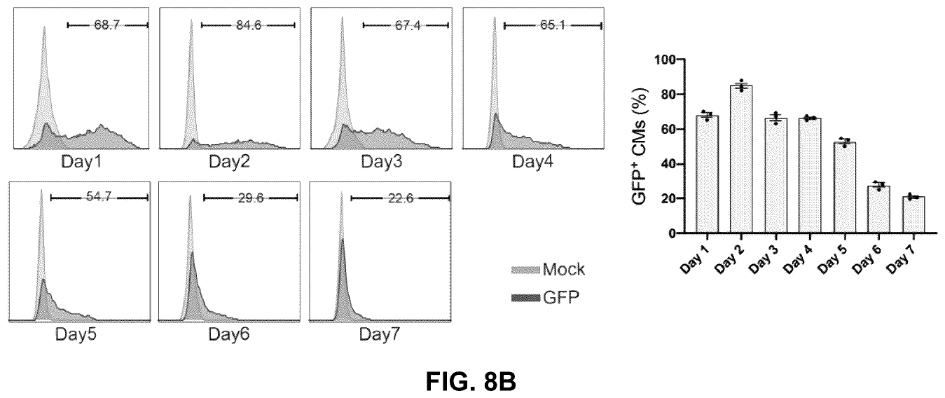

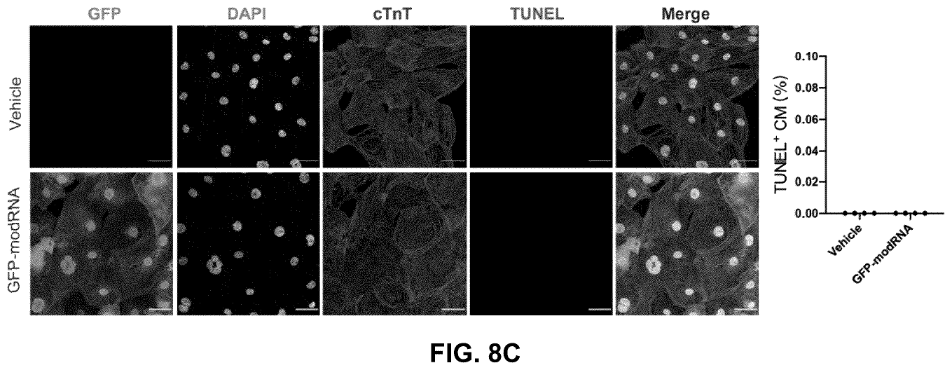

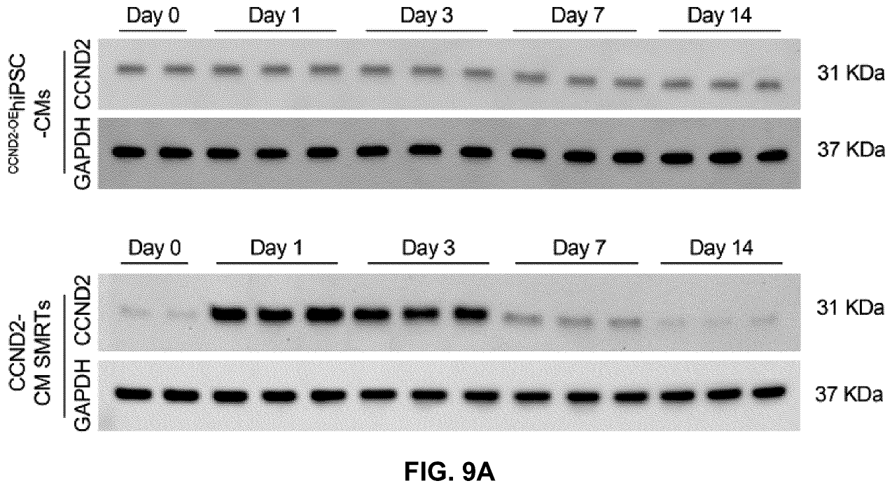

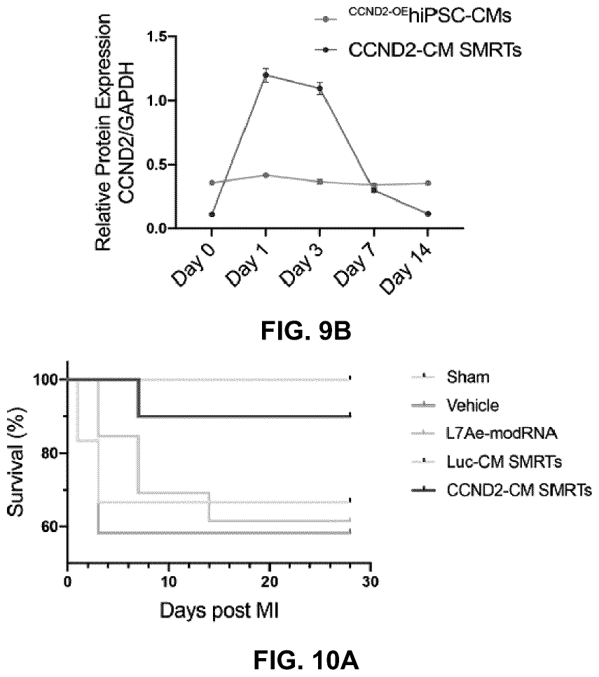

modRNAs for L7Ae, luciferase (Luc), CCND2, GFP, nuclear GFP (nGFP), and CCND2-nGFP were transcribed in vitro and validated with a bioanalyzer (FIG. 7). Subsequent experiments with GFP-modRNA indicated that 3 μg was the optimal dose for efficient modRNA translation in hiPSC-CMs (FIG. 8A), and that the GFP signal peaked 2 days after transfection before declining to 50% and 20% of maximum on days 5 and 7, respectively (FIG. 8B), and that transfection with the optimized dose did not promote hiPSC-CM apoptosis (FIG. 8C). Compared with CCND2-overexpressing hiPSC-CMs (CCND2-OEhiPSC-CMs) that have been investigated in previous reports, CCND2 protein expression level in CCND2-CM SMRTs—treated hiPSC-CMs peaked, and exceeded those in CCND2-OEhiPSC-CMs by ˜3-fold, from 1-3 days after treatment before declining to nearly undetectable levels by Day 14 (FIGS. 9A-9B). The specificity of the CM SMRTs was validated by transfecting co-cultures of hiPSC-CMs and endothelial cells (ECs) or hiPSC-CMs and fibroblasts (FBs) with 3 μg GFP-modRNA or with GFP-CM SMRTs consisting of 3 μg of GFP-modRNA and 1.5 μg of L7Ae-modRNA; robust GFP immunofluorescence was observed in all cell types after transfection with GFP-modRNA and in hiPSC-CMs that had been transfected with GFP-CM SMRTs, but not in GFP-CM SMRTs-transfected ECs or FBs (FIGS. 1B-1C).

CCND2-CM SMRTs Promotes Cardiomyocyte Proliferation and Myocardial Regeneration and Improves Recovery from AMI in Mice

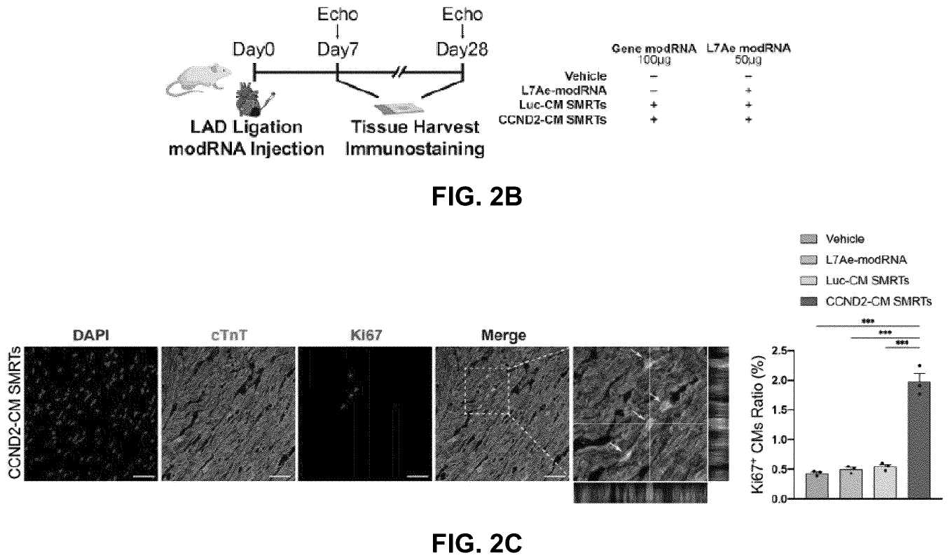

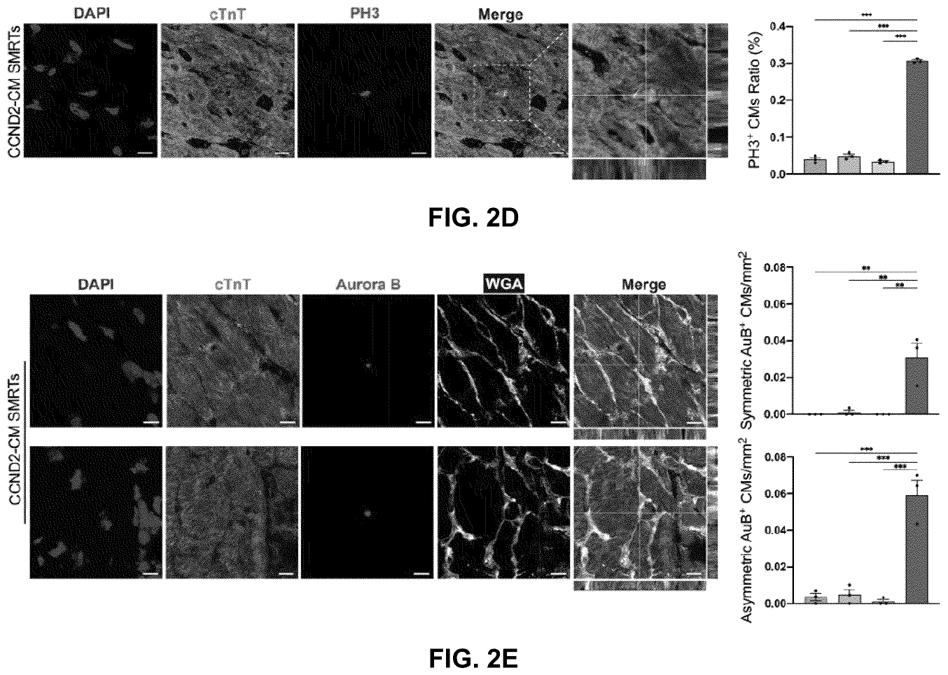

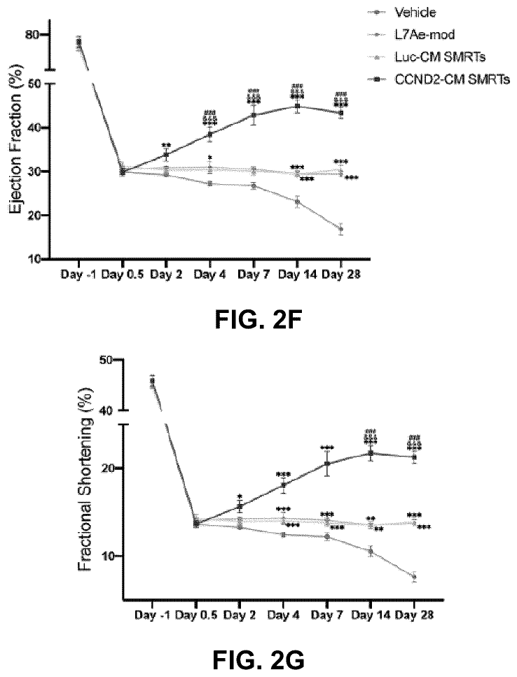

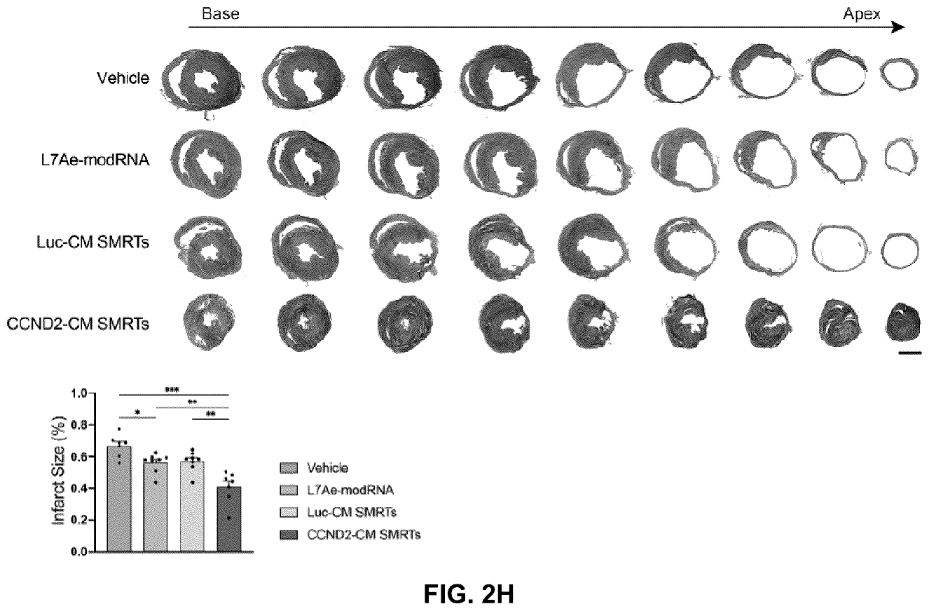

CCND2 was abundantly expressed in cultured, post-mitotic (60-day-old) hiPSC-CMs two days after transfection with CCND2-CM SMRTs (FIG. 1D), and this increase was accompanied by the upregulation of markers for proliferation (Ki67 expression; FIG. 1E), S-phase (BrdU incorporation; FIG. 1F) and the G2/M phase transition (phosphorylated histone H3 [PH3] abundance; FIG. 1G) of the cell cycle, and for cytokinesis (aurora B kinase expression; FIG. 1H). Cell division of hiPSC-CMs treated with CCND2-CM SMRTs was also evidenced by time-lapse images (FIG. 1) and cell counts (FIG. 1J). Furthermore, when GFP-CM CM SMRTs (100 μg GFP-modRNA and 50 μg L7Ae-modRNA) were intramyocardially injected into infarcted mouse hearts, immunofluorescence images of sections obtained three days later confirmed the cardiomyocyte specificity of GFP-CM SMRTs transfection in vivo: the GFP signal was observed in CM but no other cell types (FIG. 2A). Thus, whether CCND2-CM SMRTs could activate cardiomyocyte cell cycle and improve recovery from AMI was investigated by conducting experiments in a mouse AMI model (FIG. 2B).

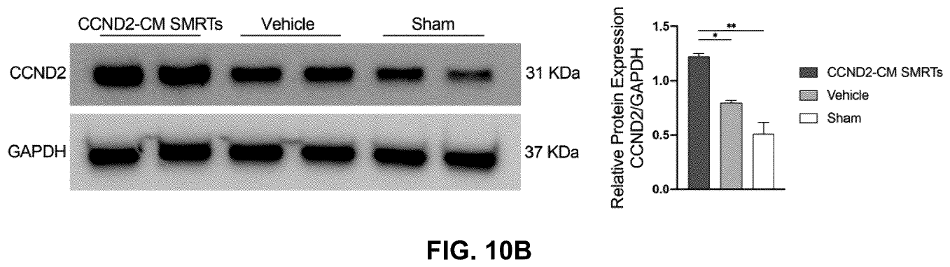

AMI was induced via permanent ligation of the left anterior descending (LAD) coronary artery, and then the animals were randomly distributed into four treatment groups. The CCND2-CM SMRTs group received 100 μg CCND2-modRNA with 50 μg L7Ae-modRNA, the Luc-CM SMRTs group received 100 μg Luc-modRNA with 50 μg L7Ae-modRNA, the L7Ae-modRNA group received 50 μg-L7Ae modRNA, and the Vehicle group received an equivalent volume of the delivery vehicle; a fifth group of animals, the Sham group, underwent all surgical procedures for MI induction except for LAD artery ligation and recovered without any of the experimental treatments. Survival rates for mice in all four groups that underwent MI were similar (FIG. 10A), and Western blot assessments conducted in myocardial tissues collected three days after MI or Sham surgery and treatment administration confirmed that CCND2 protein levels were upregulated in the hearts of animals in the CCND2-CM SMRTs group (FIG. 10B).

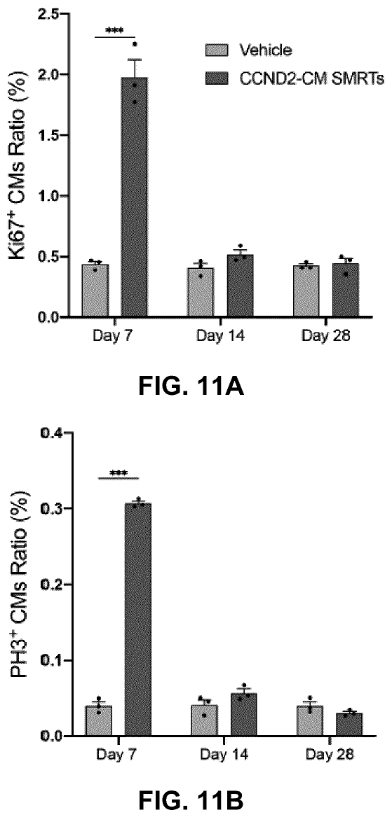

Immunofluorescence images of sections collected from the border zone of the infarct on Day 7 after MI induction and treatment indicated that the proportion of CM expressing Ki67 (FIG. 2C), PH3 (FIG. 2D), or symmetric and asymmetric AuB (FIG. 2E) was significantly higher in mice that were treated with CCND2-CM SMRTs than in the Vehicle-, L7Ae-modRNA-, or Luc-CM SMRTs— treatment groups. However, the cell cycle activity and proliferation (Ki67 and PH3) in cardiomyocytes were not significantly different between CCND2-CM SMRTs group and Vehicle group 14 days or 28 days after injection (FIGS. 11A-11B).

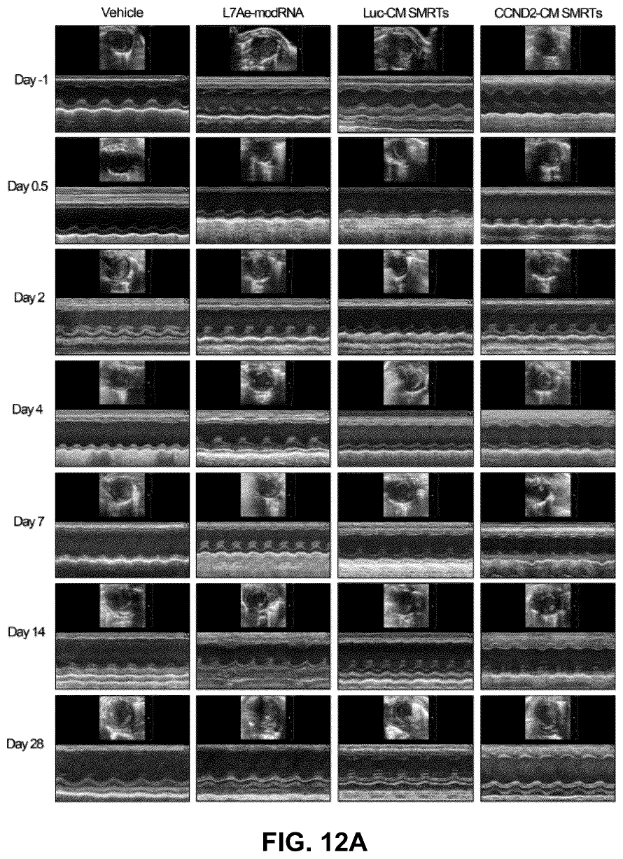

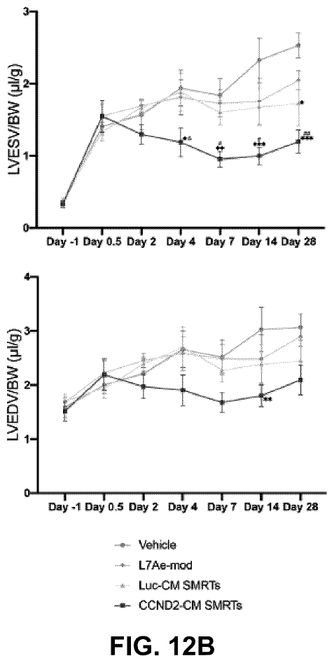

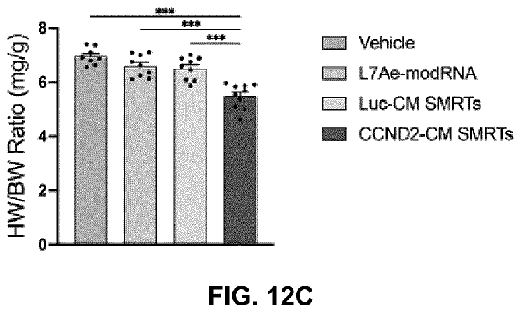

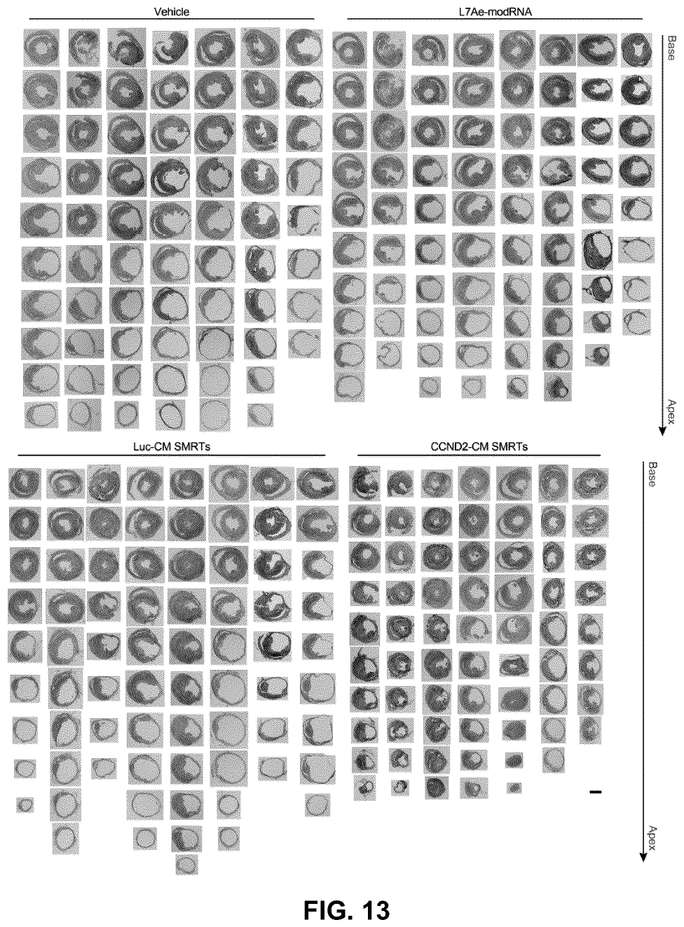

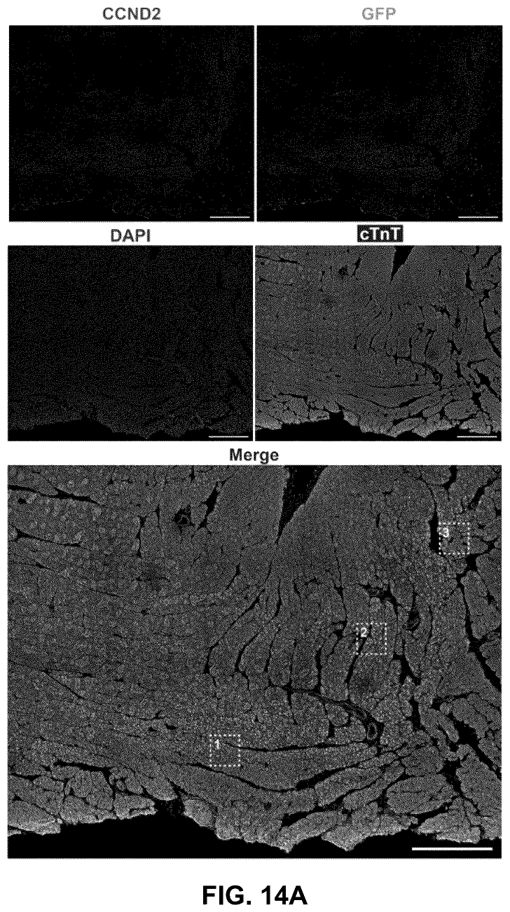

Cardiac function was evaluated 1 day before MI induction and 0.5, 2, 4, 7, 14, and 28 days afterward via echocardiographic assessments (FIG. 12A) to measure left-ventricular end-systolic volume (LVESV; FIG. 12B), end-diastolic volume (LVEDV; FIG. 12B), ejection fraction (LVEF; FIG. 2F) and fractional shortening (LVFS; FIG. 2G). Cardiac functional parameters were equivalent at Day 0.5 in all groups that underwent AMI induction, but LVEF and LVFS (FIGS. 2F-2G) progressively increased in the CCND2-CM SMRTs group through Day 14, while these measurements declined in the Vehicle group and remained stable in L7Ae-modRNA and Luc-CM SMRTs animals. Improved LV systolic and diastolic function (FIG. 12B) also indicated that CCND2-CM SMRTs could prevent the LV chamber dilatation. Measurements of cardiac fibrosis (FIGS. 2H and 13), the cross-sectional surface areas of border-zone CM (FIG. 2I), and heart-weight-to-bodyweight (HW/BW) ratios (FIG. 12C) were significantly smaller in the CCND2-CM SMRTs group than in all other injury groups. Thus, CCND2-CM SMRTs was associated with significant increases in cardiomyocyte proliferation and with significant improvements in cardiac function, infarct size, and hypertrophy when evaluated in a mouse MI model.

### CCND2-CM SMRTs Promotes Cardiomyocyte Proliferation in AMI Pig Hearts

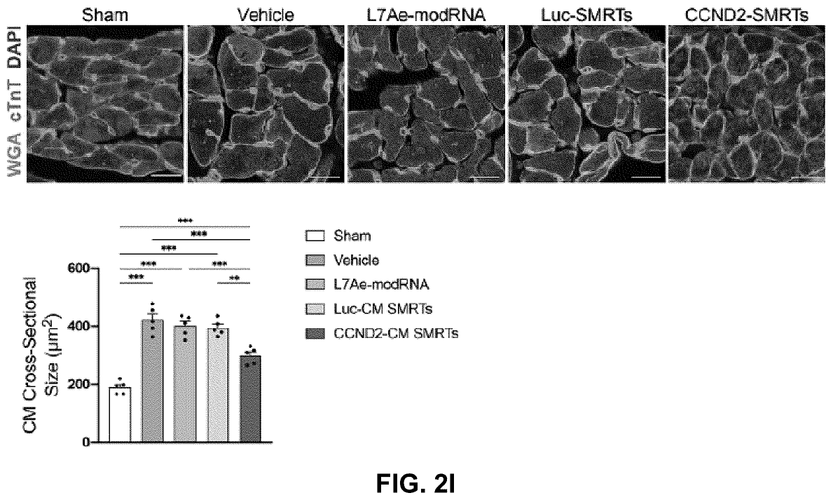

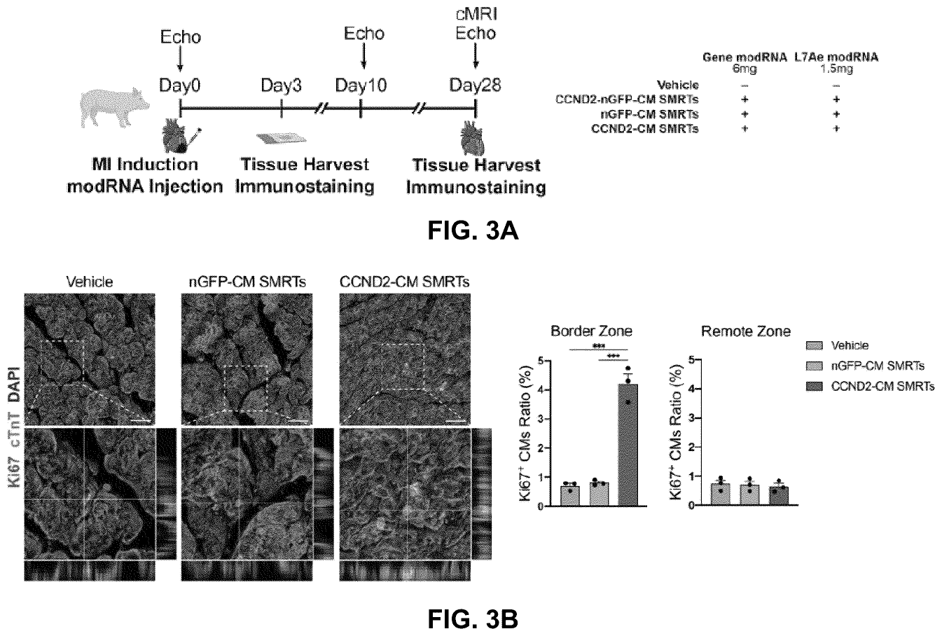

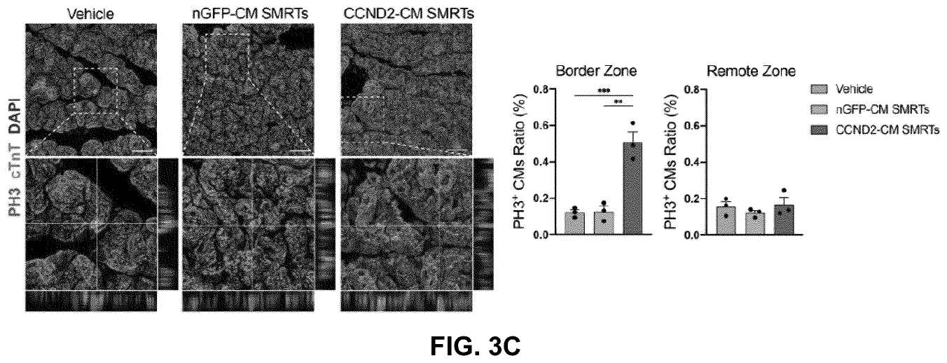

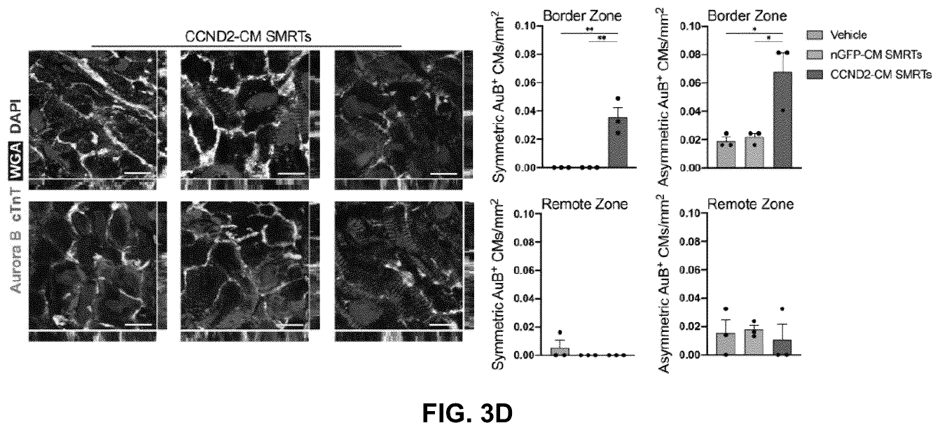

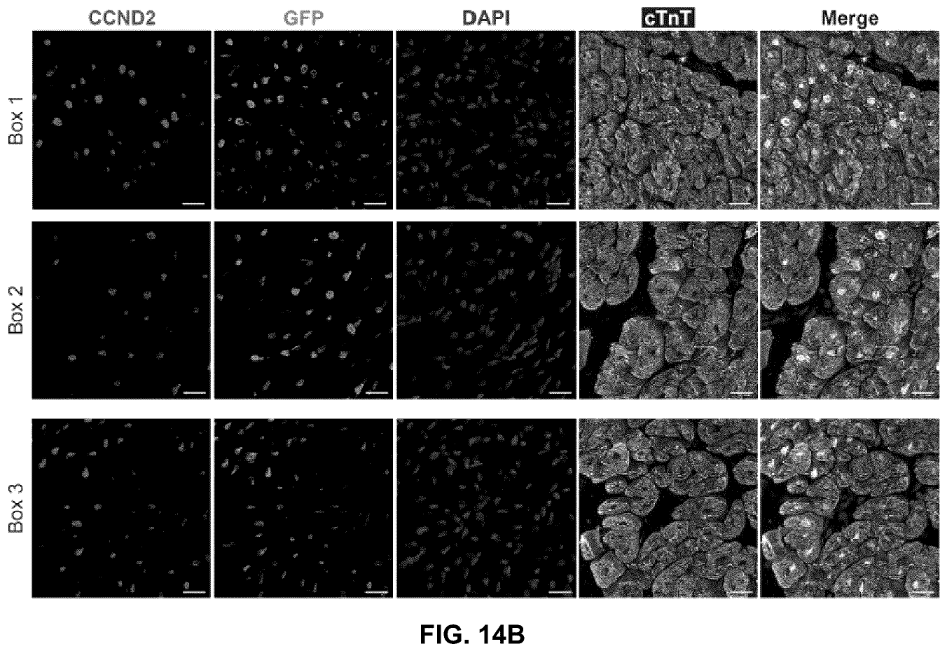

This investigation of the potency of CCND2-CM SMRTs for improving recovery from myocardial injury continued with experiments in a more clinically relevant, large-mammalian model (FIG. 3A). AMI was induced in ˜45-day-old Yorkshire pigs (body weight ˜15 Kg; of both male and female) by occluding the LAD coronary artery for 60 minutes before reperfusion, and then the animals were randomly distributed to treatments with intra-myocardial injections of 6 mg CCND2-modRNA and 1.5 mg L7Ae-modRNA (the CCND2-CM SMRTs group), 6 mg nGFP-modRNA and 1.5 mg L7Ae-modRNA (the nGFP-CM SMRTs group), or an equivalent volume of the delivery vehicle (the Vehicle group). The cardiomyocyte specificity of the CM SMRTs treatments was confirmed via immunofluorescent images of GFP expression in sections collected from infarcted pig hearts after treatment with CCND2-nGFP-modRNA and L7Ae-modRNA (FIGS. 14A-14B): CCND2 and GFP co-expression was only observed in CM.

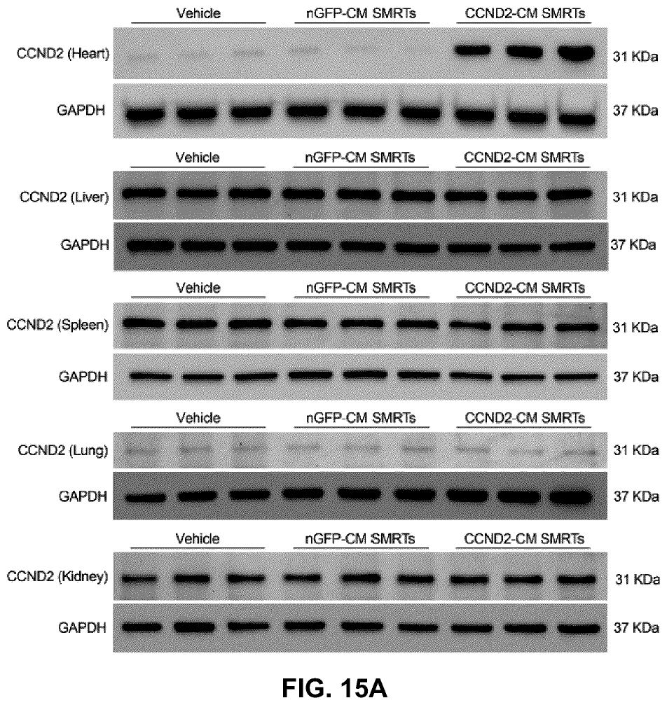

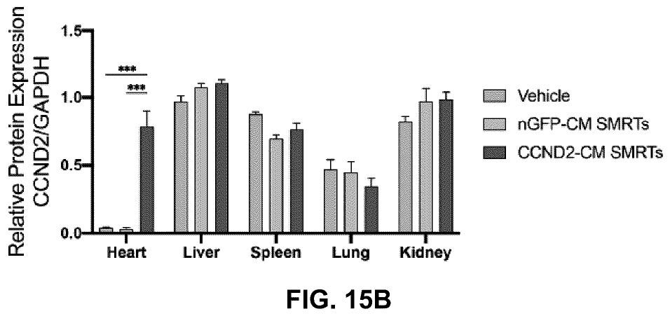

Western blot assessments conducted in organs from pigs sacrificed 3 days after MI induction and treatment confirmed that CCND2 protein levels were upregulated in the hearts of CCND2-CM SMRTs animals, but not in other major organs, and were unchanged in all organs (including the hearts) of animals in the nGFP-CM SMRTs and Vehicle groups (FIGS. 15A-15B). Notably, the CM of pigs continued to undergo multinucleation events (i.e., mitosis without cytokinesis) for up to six months after birth; thus, some residual evidence of Ki67 expression, PH3 abundance and asymmetric AuB expression was observed in the border zones of nGFP-CM SMRTs- and Vehicle-treated animals, as well as in the remote (uninjured) zones of animals in all three groups. However, Ki67- and PH3-positive border-zone CM were significantly more common in hearts from CCND2-CM SMRTs animals than from the nGFP-CM SMRTs and Vehicle treatment groups (Ki67: 5.99-fold increase, p<0.001 relative to Vehicle; PH3: 4.17-fold increase, p<0.001 relative to Vehicle; FIGS. 3C-3D). Karyokinesis marker asymmetric AuB was expressed in all cardiac tissue and elevated by CCND2-CM SMRTs, whereas the cytokinesis marker symmetric AuB was expressed in border-zone CM of CCND2-CM SMRTs-treated hearts, symmetric AuB-positive CM were essentially absent in the border zones of hearts treated with nGFP-CM SMRTs or the delivery Vehicle and in the remote zones of hearts from all three groups (FIG. 3E).

CCND2-CM SMRTs Reduces Infarct Size and Improves Global and Regional Cardiac Function after AMI in Pigs

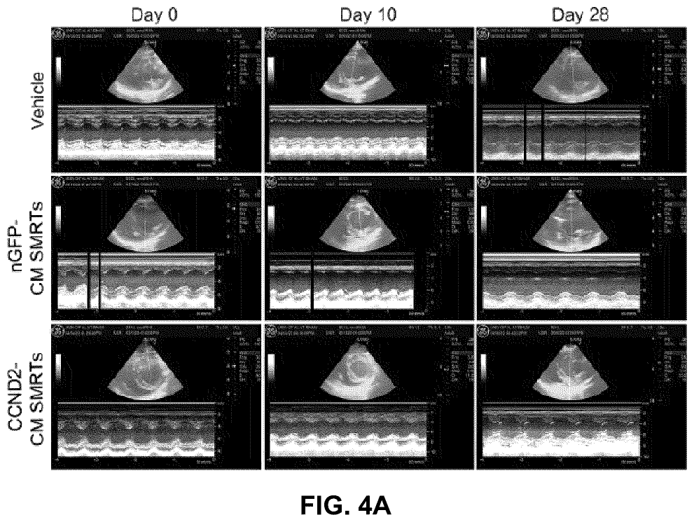

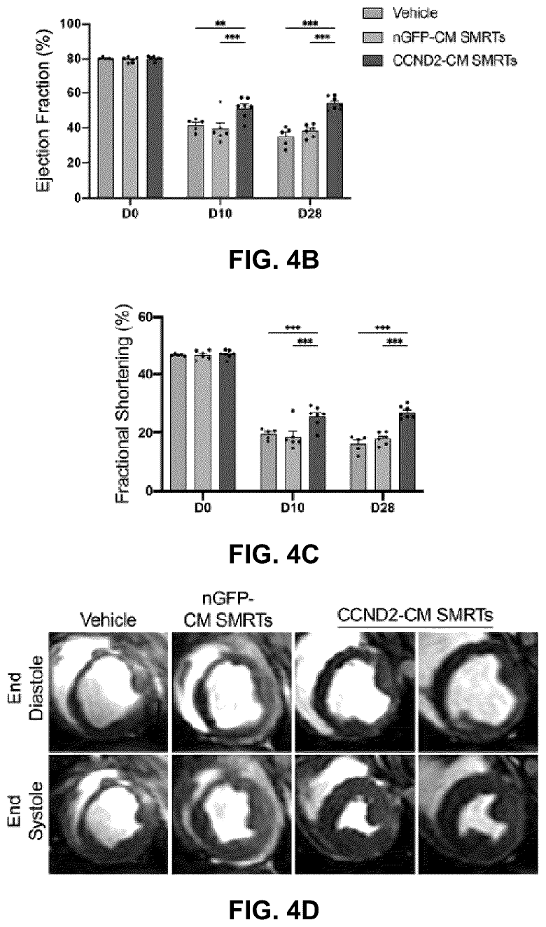

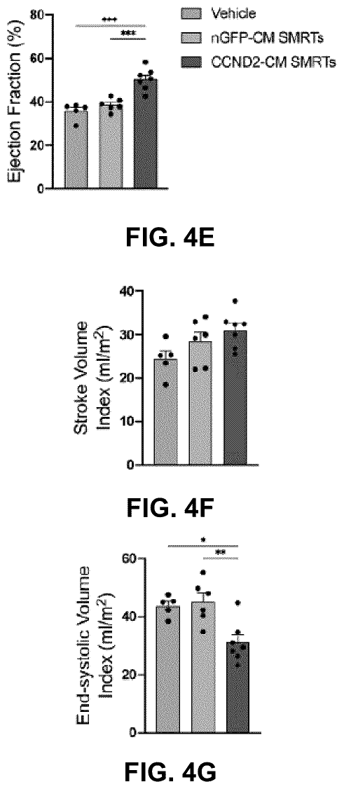

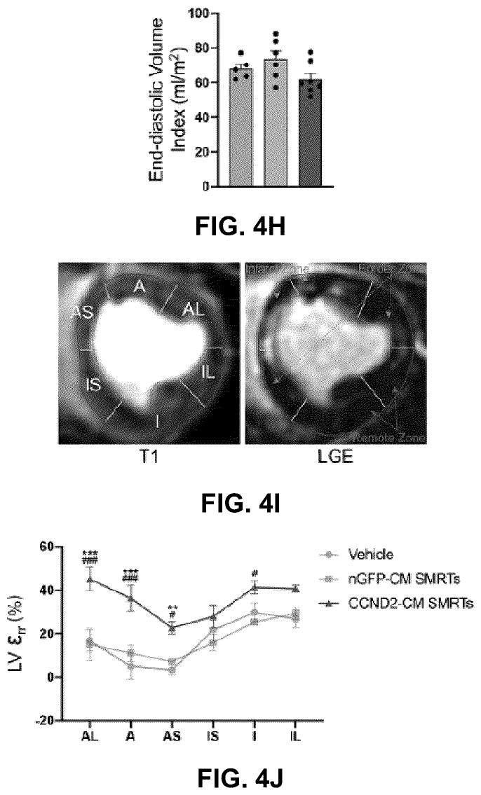

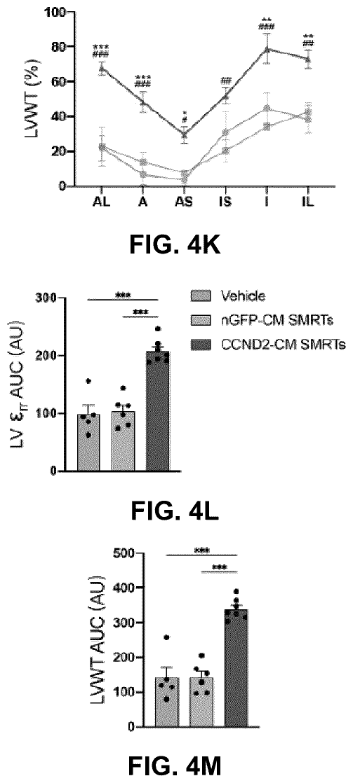

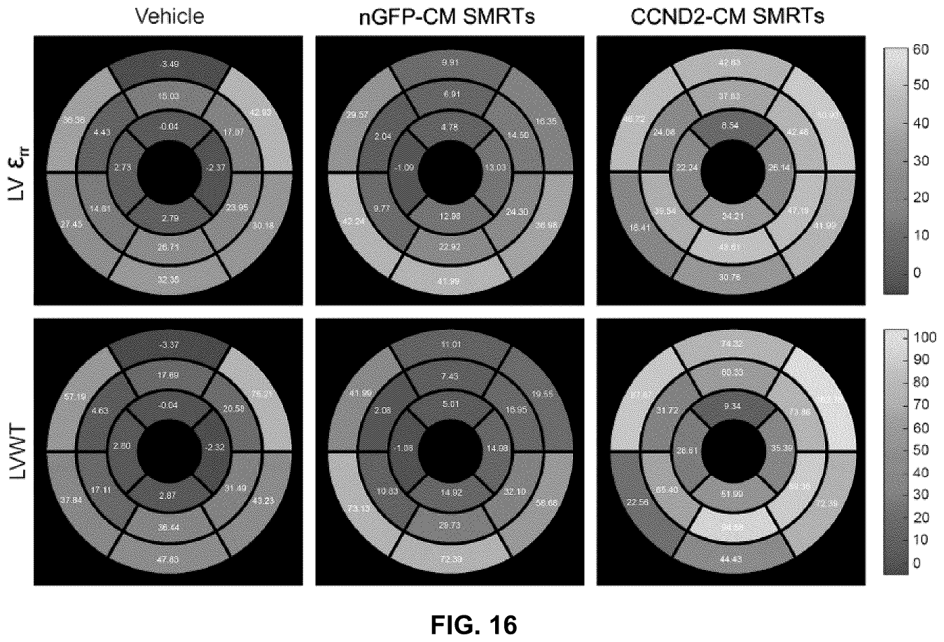

Cardiac function was evaluated before AMI induction (Day 0) and 10 and 28 days afterward via echocardiography (FIG. 4A) and on Day 28 via cardiac magnetic resonance imaging (cMRI) (FIG. 4D). Echocardiographic measurements of LVEF (FIG. 4B) and LVFS (FIG. 4C) were equivalent in all three treatment groups on Day 0 and significantly greater in animals treated with CCND2-CM SMRTs than in nGFP-SMRT- or Vehicle-treated animals on Day 10 and Day 28; measurements appeared to increase from Day 10 to Day 28 in animals from the CCND2-SMRT group while measurements in the Vehicle-treatment group declined, but the differences between the two time points were not significant. cMRI assessments on Day 28 also indicated that LVEF was significantly greater (1.41-fold increase, p<0.001 relative to Vehicle; FIG. 4E). In CCND2-CM SMRTs-treated animals than in either of the other two treatment groups, and that the increases were primarily attributable to improvements in end-systolic (FIG. 4G), rather than end-diastolic (FIG. 4H), volume. Furthermore, when cMRI assessments (FIG. 4I) were used to calculate radial strain (εRR) and LV wall thickening (LVWT) in accordance with the American Heart Association's 17-segment model (FIG. 16), the most significant regional contractility improvement occurred in the anterolateral and anterior walls of CCND2-SMRT-treated animals than in the nGFP-SMRT or Vehicle treatment groups (FIGS. 4J-4K). The area under the curve (AUC) of both parameters was then calculated, showing significant recovery of both εRR and LVWT in the CCND2-SMRT group 28 days after MI (FIGS. 4L-4M).

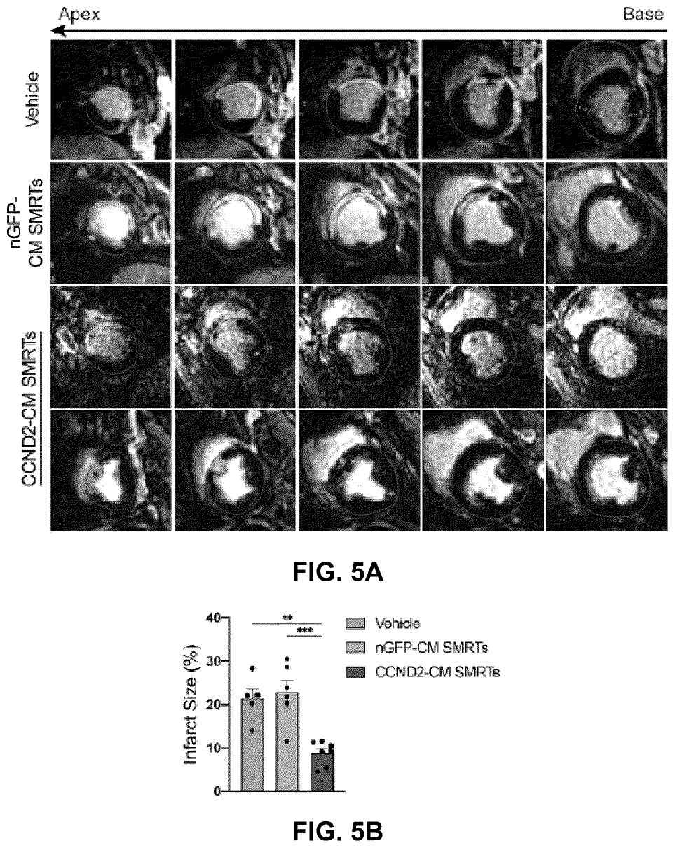

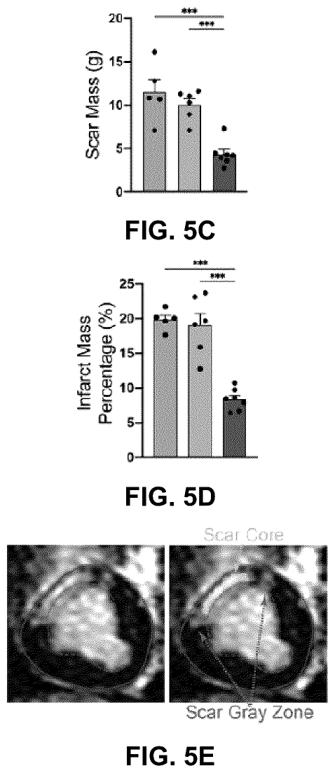

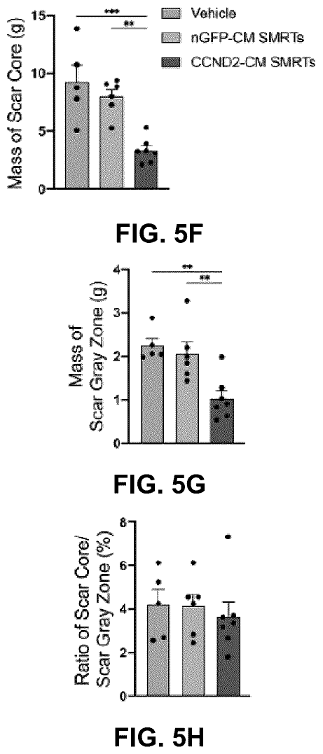

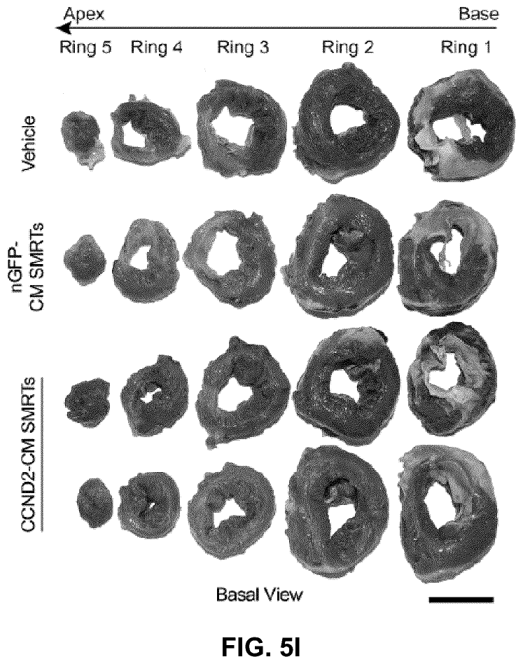

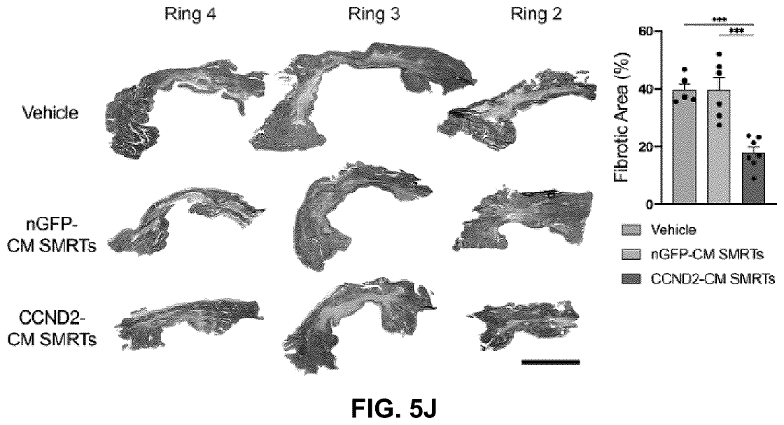

Infarct size was evaluated on Day 28 via late gadolinium enhancement (LGE) cMRI. The gadolinium-retaining region (FIG. 5A) was noticeably smaller in CCND2-CM SMRTs-treated hearts than in hearts from animals in the nGFP-CM SMRTs- or Vehicle-treatment groups, and calculated values for infarct size (proportion of the LV surface area that was occupied by the infarct; FIG. 5B; 8.80±1.07% vs. 21.38±2.31%, CCND2-CM SMRTs vs. Vehicle; p<0.01), infarct mass (FIG. 5C), and infarct mass percentage (the ratio of infarct mass relative to LV weight; FIG. 5D) were significantly smaller (by more than ˜50%, p<0.01) in pigs treated with CCND2-CM SMRTs than in either of the other two groups. The mass of the infarct core (FIG. 5F) and the gray zone (i.e., the region of both necrotic and viable myocardium surrounding the infarct core) (FIG. 5G) were also significantly smaller in CCND2-CM SMRTs-treated hearts than in hearts from the nGFP-CM SMRTs- or Vehicle-treatment groups (infarct core decreased by 64.07%, p<0.001 relative to Vehicle; infarct gray zone decreased by 54.23%, p<0.01 relative to Vehicle), and the results from LGE-cMRI measurements were consistent with observations in serial sections of the LVs from pigs in each of the three treatment groups (FIGS. 5I-5J): the fibrotic area was significantly smaller in sections from CCND2-CM SMRTs-treated animals (FIG. 5J).

CCND2-CM SMRTs does not Induce Long-Term Cardiomyocyte Proliferation or Increase the Risk of Arrythmia after Administration to Infarcted Pig Hearts

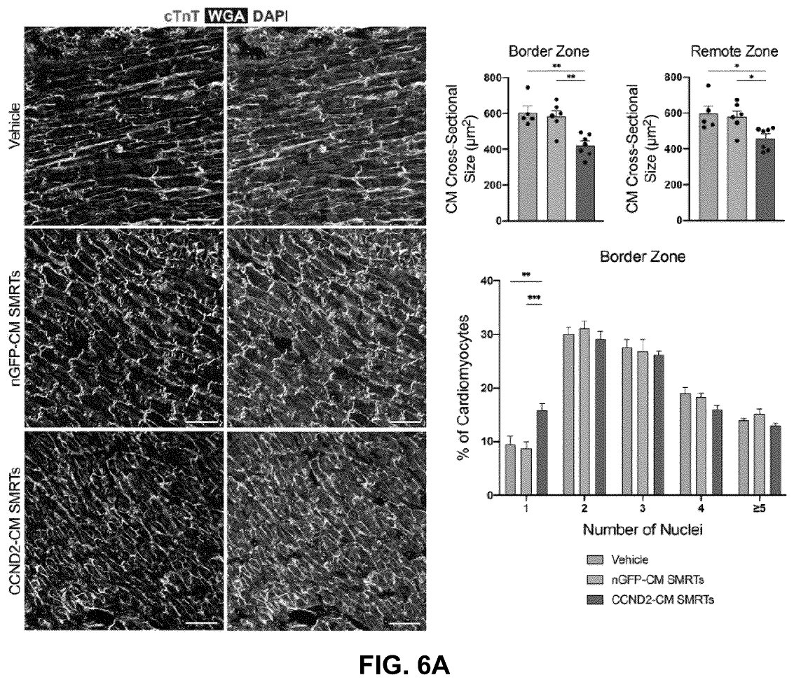

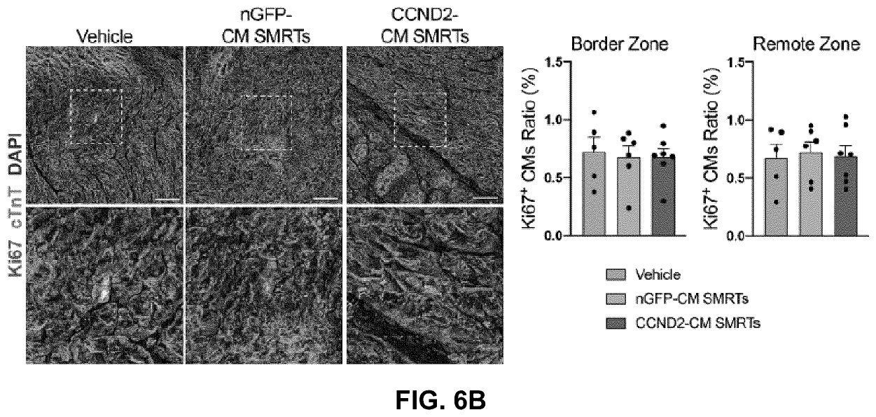

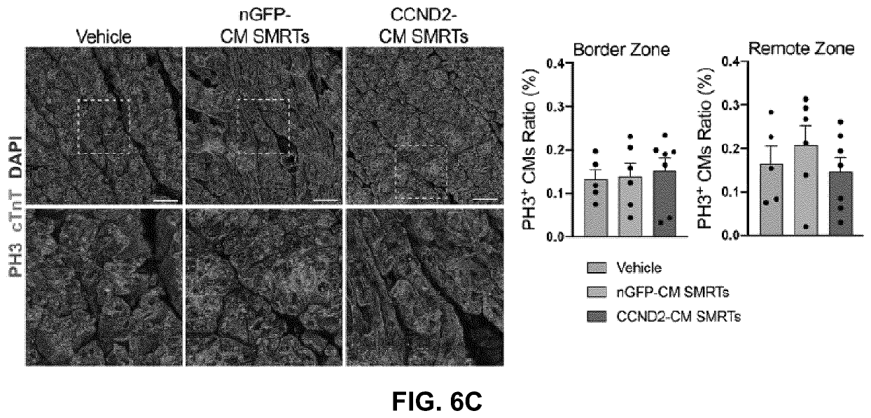

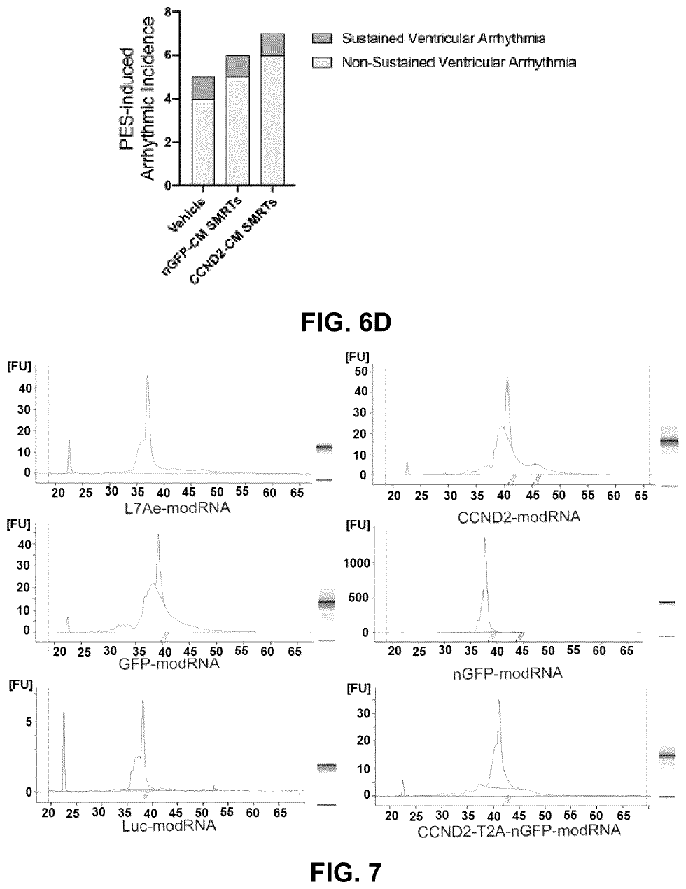

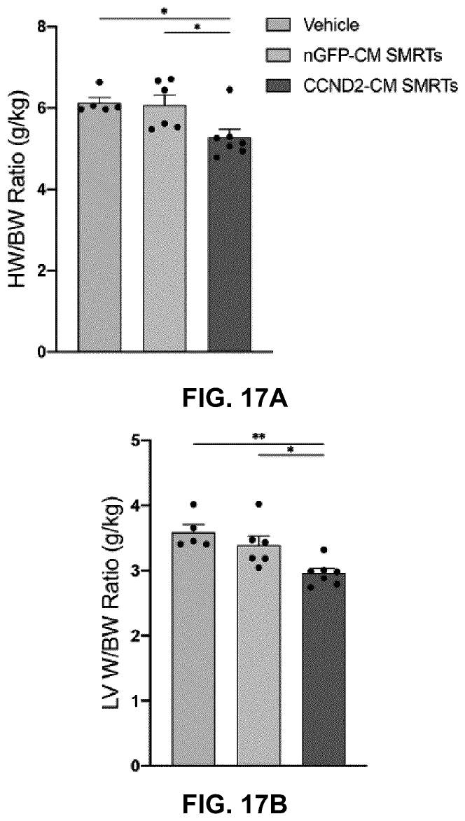

At 4 weeks after the surgery, cardiac hypertrophy was significantly reduced in CCND2-CM SMRTs group evidenced by decreased heart weight/body weight ratio and left ventricular weight/body weight ratio (FIGS. 17A-17B). Histologically, the cross-sectional surface areas of the border zone and remote zone cardiomyocytes were evaluated by immunostaining and showed that CCND2-CM SMRTs significantly decreased CM cross-sectional surface areas as compared with those from Vehicle- and nGFP-CM SMRTs¬-treated hearts (FIG. 6A), suggesting CCND2-CM SMRTs were able to protect the entire left ventricle from MI-related hypertrophy. The percentage of mono-, bi-, and multi-nucleated CMs in border zone was also calculated to evaluate CM division (FIG. 6B). The number of mononucleated cardiomyocytes was higher in CCND2-CM SMRTs¬-treated hearts than in control hearts (FIG. 6B), providing further support that cardiomyocytes were dividing in CCND2-CM SMRTs-treated hearts.

One of the primary safety concerns associated with the administration of CCND2-overexpressing hiPSC-CMs is that long-term, uncontrolled expression of cell-cycle regulatory molecules could lead to excessive cardiomyocyte proliferation and arrhythmia. However, Ki67- and PH3-positive border-zone and remote-zone CM were no more prevalent on Day 28 in CCND2-CM SMRTs-treated hearts than in hearts from animals in the nGFP-CM SMRTs- or Vehicle-treatment groups (FIGS. 6C-6D). Furthermore, when the hearts of pigs were paced via programmed electrical stimulation (PES), incidents of ventricular tachycardia and fibrillation were equally common in animals from all three treatment groups (FIG. 6E). Thus, intramyocardial CCND2-CM SMRTs administration produced only a transient increase in cardiomyocyte proliferation and was not associated with an elevated risk of arrhythmogenic complications.

### Example 3: Discussion and Conclusions

Despite current intensive treatment regimens, patients with severe AMI often progress to end stage CHF, which is one of the most significant problems in public health today. From the molecular and cellular perspective, heart failure occurs due to the loss of the contractile unit of the left ventricle: CM. However, the regenerative capacity of adult mammalian hearts is limited because the majority of CM exit the cell cycle shortly after birth and arrest at the G1/S transition, known as the restriction point (R-point) of cell cycle withdrawal. R-point transit is partially governed by the activity of cyclin-dependent kinase 4 (CDK4) and its obligate cofactors, the D-type cyclins. CDK4/cyclin D complex disrupts RB-E2F binding by phosphorylating the RB protein family, permitting E2F-mediated transcription of genes involved in activating DNA synthesis and cell cycle progression. Previous study on the cyclin D family using transgenic mouse model has demonstrated that cyclin D2 gene driven by a CM-restricted MHC promoter led to a robust nuclear CCND2 overexpression and preserved CM cell cycle activity in both healthy and myocardial hypertrophic mice. Whereas when hypertrophy was induced in cyclin D1 or D3 transgenic mice, the nuclear localization of cyclin D1 and D3 protein was compromised, impeding their association with CDK4 before nuclear translocation thus, failed to maintain DNA synthesis under myocardial hypertrophy.

In the present study, the immunostaining results from both the in vitro (FIG. 1D) and in vivo (FIGS. 14A-14B) experiments all indicate the strong CCND2 expression localized in CM nuclear, facilitating the following R-point transition. However, although the efficiency of modRNA transfection is exceptionally high (˜85% in vivo), it is not perfect, so a few non-cardiomyocytes may express GFP because they were transfected with nGFP-modRNA but not L7Ae-modRNA. Following R-point transition induced by CCND2, enhanced activity of DNA synthesis (FIG. 1F) and cell cycle progression (including G2/M transition and cytokinesis; FIGS. 1E and 1G-1H; FIGS. 2C-2E; FIGS. 3B-3D), and increased cell division (FIGS. 1I-1J and 6B) of CM were observed both in vitro and in vivo, which in turn, are accompanied by a significant decrease of infarction size (FIGS. 2H, 5A-5J and 13) and restore cardiac function (FIGS. 2F-2G, 4A-4M, and 12A-12B).

The increase in the proportion of cardiomyocytes that expressed PH3 and AuB in the hearts of animals treated with CCND2-CM SMRTs after MI is consistent, or even greater, than the increases reported in other studies of induced cardiomyocyte proliferation. Nevertheless, it is acknowledged that PH3+ and AuB+ cardiomyocytes may be undergoing either multinucleation events (i.e., karyokinesis) or cytokinesis, so the analysis has been refined by determining the proportion of cardiomyocytes in which AuB expression is localized asymmetrically, which identifies a karyokinesis event within a single cell, or symmetrically between two daughter nuclei. Mouse (FIG. 2E) and pig data (FIG. 3D) suggest that CCND2-CM SMRTs could increase karyokinesis and reactivate cytokinesis activities in both species. The pigs used in this investigation were juvenile animals, so the cardiomyocytes were predominantly bi- or tetranuclear, but still included a sizable number (˜10%) of mononuclear cells that were believed to be the primary source of the proliferative activity observed in these studies (FIG. 6B).

These findings of CCND2 preventing cardiomyocytes hypertrophy (FIGS. 2I, 6A, 12C, and 17A-17B) differ from several previous studies which demonstrate that cardiomyocyte CycD2 expression increases in response to pressure overload or treatment with angiotensin II, and that cardiomyocyte-specific ablation of CycD2 inhibits the hypertrophy observed in transgenic mice that overexpress cMyc. Importantly, the models used in these investigations are fundamentally different than the MI models used in these experiments, because they were specifically designed to induce cardiac hypertrophy. Furthermore, although the previous reports convincingly demonstrated that CycD2 is required for the hypertrophic growth of cardiomyocytes in these models, they did not demonstrate that hypertrophy can be induced via CycD2 overexpression alone and, consequently, do not conflict with the observation that both cardiomyocyte cross-sectional surface areas (FIGS. 2I and 6A) and heart weight/body weight ratios (FIGS. 12C and 17A-17B) were lower in the CCND2-CM SMRTs groups than in Vehicle-treated animals. It is believed that MI-induced cardiac hypertrophy is caused by the loss of functional contractile tissue, which increases the physiological demand on surviving cardiomyocytes, and that the CCND2-CM SMRTs alleviates this burden both by reducing the number of pre-existing cardiomyocytes that undergo apoptosis and by promoting the generation of new cardiomyocytes.

A major concern on some of the established approaches for CM regeneration is that excessive activation of CM and reduction in sarcomere stability may lead to lethal arrhythmia. For instance, a previous study offered an approach by transplanting CCND2 overexpressed hiPSC-CMs into myocardial infarcted porcine heart, which enhanced the proliferation of both the transplanted and endogenous CMs22. Although no arrhythmic incidence was observed, a concern has been raised that the lentiviral transduced CCND2 may lead to permanent and prolonged cell cycle activation and excessive CM proliferation. For another instance, lethal arrhythmic incidence is reported by a recent study where AAV-mediated delivery of miR-199a was evaluated in a pig MI model, the miR-199a treatment led to persistent miR-199a expression, and the majority of pigs died from sudden arrhythmia despite improvements in contractility and scar size. In order to minimize the arrhythmogenic complications caused by persistent CM proliferation, it is aimed to induce a transient cell cycle activation in CM by delivering the targeted gene, CCND2, using CM SMRTs technology, taking advantage of its controlled and bell-shaped expression profile.

The successful use of modRNA technology for the development of vaccines against SARS-CoV-2 demonstrates the feasibility of this platform for administration to patients, and because modRNA transcripts are expressed only transiently, they are unlikely to be associated with some of the long-term safety concerns that have limited the clinical translation of other delivery methods. Cell-type specificity, into CM, is also a key component of strategies for improving myocardial repair via the delivery of cell-cycle regulators to the heart, because off-target expression of these molecules in other cell types could promote fibrosis, inflammation, and other complications. Here, it is demonstrated that the CM SMRTs platform can be used to rapidly upregulate CCND2 expression in CM nuclear with exceptional cell-type specificity.

CCND2-CM SMRTs induced transient CM cell cycle activation, but did not increased the risk of arrhythmia. It was observed that CCND2 expression in cultured CM peaked 1 day after the cells were treated with CCND2-CM SMRTs and declined to nearly undetectable levels by Day 14 (FIGS. 9A-9B), which is largely consistent with previous reports that indicated modRNA expression endures for 8-12 days in mice. Although the duration of CM SMRTs-driven CCND2 expression was not thoroughly characterized in-vivo, reactivated CM turnover by CCND2-CM SMRTs dropped to background level 28 days after treatment administration as Ki67- and PH3-positive border-zone CM were not more common in pigs treated with CCND2-CM SMRTs than in Vehicle-treated pigs (FIGS. 6C-6D). Importantly, no spontaneous arrhythmia induced lethality was observed in pigs during the 4-week follow-up period, and the PES-induced sustained ventricular arrhythmia was equally common among all 3 groups (FIG. 6E), demonstrating the safe profile of CCND2-CM SMRTs partially contributed by the transient reactivation of CM cell cycle. The decreased infarct gray zone also contributed partially to the limited arrhythmic incidence in this study (FIG. 5G), as the heterogeneous mixture of viable and nonviable myocardium increases the probability of electric circuit reentries.

It was also observed that L7Ae-modRNA alone protected cardiomyocyte apoptosis. Both miR-1 and miR-208 contribute to cardiomyocyte apoptosis and were likely to be “sponged” by miR1 and miR208 recognition sites on the L7Ae modRNA, as shown previously. Thus, since modRNA transcripts appear to be expressed almost immediately after transfection, at least some of the improvements observed in CCND2-CM SMRTs—treated animals could depend on the time of treatment administration (i.e., immediately after LAD artery ligation or reperfusion), which likely reduced the number of CM that were lost to the infarct event.

This study has some limitations. It is noted that interventions immediately after the AMI has limitations from the perspectives of translatability and may have confounding factors such as post-conditioning effects. However, it is appreciated that almost all preclinical animal models have limitations considering extrapolating findings to patients with chronic postinfarction LV remodeling and heart failure. In the current study the main hypothesis of whether the modRNA technology, specifically the SMRTs intervention, can reactive CM cell-cycle in large mammal hearts with AMI, and consequently decrease of infarct size and prevent LV dilatation, is investigated.

In conclusion, the results presented in this report demonstrate that CM SMRTs could rapidly and transiently drive the expression of CCND2 in CM nuclear with high cell-type specificity, and that intramyocardial injections of CCND2-CM SMRTs activated cardiomyocyte cell cycle, reduced infarct size, and improved cardiac performance in both small and large mammalian models of myocardial injury. These findings demonstrate, for the first time, that an acute myocardial infarct of mammalian hearts could be remuscularized by the CM modRNA SMRTs technology.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Abouleisa R R E, et al. Transient Cell Cycle Induction in Cardiomyocytes to Treat Subacute Ischemic Heart Failure. Circulation. 2022; 145:1339-1355.
2. Alvarez R, Jr., et al. Cardiomyocyte cell cycle dynamics and proliferation revealed through cardiac-specific transgenesis of fluorescent ubiquitinated cell cycle indicator (FUCCI). J Mol Cell Cardiol. 2019; 127:154-164.
3. Bergmann O, et al. Dynamics of Cell Generation and Turnover in the Human Heart. Cell. 2015; 161:1566-75.
4. Busk P K, et al. Involvement of cyclin D activity in left ventricle hypertrophy in vivo and in vitro. Cardiovasc Res. 2002; 56:64-75.
5. Carlsson L, et al. Biocompatible, Purified VEGF-A mRNA Improves Cardiac Function after Intracardiac Injection 1 Week Post-myocardial Infarction in Swine. Mol Ther Methods Clin Dev. 2018; 9:330-346.
6. Cerqueira M D, et al, American Heart Association Writing Group on Myocardial S and Registration for Cardiac I. Standardized myocardial segmentation and nomenclature for tomographic imaging of the heart. A statement for healthcare professionals from the Cardiac Imaging Committee of the Council on Clinical Cardiology of the American Heart Association. Circulation. 2002; 105:539-42.
7. Chen W, et al. TT-10-loaded nanoparticles promote cardiomyocyte proliferation and cardiac repair in a mouse model of myocardial infarction. JCI Insight. 2021; 6.
8. Dowdy S F, et al. Physical interaction of the retinoblastoma protein with human D cyclins. Cell. 1993; 73:499-511.
9. Engel F B, et al. p38 MAP kinase inhibition enables proliferation of adult mammalian cardiomyocytes. Genes Dev. 2005; 19:1175-87.
10. Eulalio A, et al. Functional screening identifies miRNAs inducing cardiac regeneration. Nature. 2012; 492:376-381.
11. Fan C, et al. Cardiomyocytes from CCND2-overexpressing human induced-pluripotent stem cells repopulate the myocardial scar in mice: A 6-month study. J Mol Cell Cardiol. 2019; 137:25-33.
12. Fukuda K, et al. Angiotensin II potentiates DNA synthesis in AT-1 transformed cardiomyocytes. J Mol Cell Cardiol. 1998; 30:2069-80.
13. Gabisonia K, et al. MicroRNA therapy stimulates uncontrolled cardiac repair after myocardial infarction in pigs. Nature. 2019; 569:418-422.
14. Gabisonia K, et al. MicroRNA therapy stimulates uncontrolled cardiac repair after myocardial infarction in pigs. Nature. 2019; 569:418-422.
15. Gao L, et al. Exosomes secreted by hiPSC-derived cardiac cells improve recovery from myocardial infarction in swine. Sci Transl Med. 2020; 12.
16. Gao L, et al. Large Cardiac Muscle Patches Engineered From Human Induced-Pluripotent Stem Cell-Derived Cardiac Cells Improve Recovery From Myocardial Infarction in Swine. Circulation. 2018; 137:1712-1730.
17. Gao L, et al. Myocardial tissue engineering with cells derived from human-induced pluripotent stem cells and a native-like, high-resolution, 3-dimensionally printed scaffold. Circulation research. 2017; 120:1318-1325.
18. Hadas Y, et al. Altering Sphingolipid Metabolism Attenuates Cell Death and Inflammatory Response After Myocardial Infarction. Circulation. 2020; 141:916-930.
19. Hadas Y, et al. Optimizing Modified mRNA In Vitro Synthesis Protocol for Heart Gene Therapy. Mol Ther Methods Clin Dev. 2019; 14:300-305.
20. Hamma T, et al. Structure of protein L7Ae bound to a K-turn derived from an archaeal box H/ACA sRNA at 1.8 A resolution. Structure. 2004; 12:893-903.
21. Hassink R J, et al. Cardiomyocyte cell cycle activation improves cardiac function after myocardial infarction. Cardiovasc Res. 2008; 78:18-25.
22. Haubner B J, et al. Functional Recovery of a Human Neonatal Heart After Severe Myocardial Infarction. Circ Res. 2016; 118:216-21.
23. Henderson J M, et al. Cap 1 Messenger RNA Synthesis with Co-transcriptional CleanCap((R)) Analog by In Vitro Transcription. Curr Protoc. 2021; 1:e39.
24. Hesse M, et al. Midbody Positioning and Distance Between Daughter Nuclei Enable Unequivocal Identification of Cardiomyocyte Cell Division in Mice. Circ Res. 2018; 123:1039-1052.
25. Itoh T, et al. Body surface area measurement in juvenile miniature pigs using a computed tomography scanner. Exp Anim. 2017; 66:229-233.
26. Kaur K, et al. Direct Reprogramming Induces Vascular Regeneration Post Muscle Ischemic Injury. Mol Ther. 2021.
27. Kondrat J, et al Synthesis of Modified mRNA for Myocardial Delivery. Methods Mol Biol. 2017; 1521:127-138.
28. Leach J P, et al. Hippo pathway deficiency reverses systolic heart failure after infarction. Nature. 2017; 550:260-264.
29. Li J M, et al. Role of G1 phase cyclins and cyclin-dependent kinases during cardiomyocyte hypertrophic growth in rats. Am J Physiol. 1998; 275:H814-22.
30. Lian X, et al. Robust cardiomyocyte differentiation from human pluripotent stem cells via temporal modulation of canonical Wnt signaling. Proc Natl Acad Sci USA. 2012; 109:E1848-57.
31. Liu S, et al. Gene therapy knockdown of Hippo signaling induces cardiomyocyte renewal in pigs after myocardial infarction. Sci Transl Med. 2021; 13.
32. Magadum A, et al. Pkm2 Regulates Cardiomyocyte Cell Cycle and Promotes Cardiac Regeneration. Circulation. 2020; 141:1249-1265.
33. Magadum A, et al. Specific Modified mRNA Translation System. Circulation. 2020; 142:2485-2488.
34. Magadum A, et al. Therapeutic Delivery of Pip4k2c-Modified mRNA Attenuates Cardiac Hypertrophy and Fibrosis in the Failing Heart. Adv Sci (Weinh). 2021; 8:2004661.
35. Mohamed T M A, et al. Regulation of Cell Cycle to Stimulate Adult Cardiomyocyte Proliferation and Cardiac Regeneration. Cell. 2018; 173:104-116.e12.
36. Nakada Y, et al. Single Nucleus Transcriptomics: Apical Resection in Newborn Pigs Extends the Time Window of Cardiomyocyte Proliferation and Myocardial Regeneration. Circulation. 2022; 145:1744-1747.
37. Nakamura Y, et al. Xenotransplantation of long-term-cultured swine bone marrow-derived mesenchymal stem cells. Stem Cells. 2007; 25:612-20.
38. Nguyen N U N, et al. A calcineurin-Hoxb13 axis regulates growth mode of mammalian cardiomyocytes. Nature. 2020; 582:271-276.
39. Pasumarthi K B, et al. Targeted expression of cyclin D2 results in cardiomyocyte DNA synthesis and infarct regression in transgenic mice. Circ Res. 2005; 96:110-8.
40. Polacin M, et al. Segmental strain analysis for the detection of chronic ischemic scars in non-contrast cardiac MRI cine images. Sci Rep. 2021; 11:12376.
41. Porrello E R, et al. Regulation of neonatal and adult mammalian heart regeneration by the miR-15 family. Proc Natl Acad Sci USA. 2013; 110:187-92.
42. Porrello E R, et al. Transient regenerative potential of the neonatal mouse heart. Science. 2011; 331:1078-80.
43. Rurik J G, et al. CAR T cells produced in vivo to treat cardiac injury. Science. 2022; 375:91-96.
44. Schmidt A, et al. Infarct tissue heterogeneity by magnetic resonance imaging identifies enhanced cardiac arrhythmia susceptibility in patients with left ventricular dysfunction. Circulation. 2007; 115:2006-14.
45. Senyo S E, et al. Mammalian heart renewal by pre-existing cardiomyocytes. Nature. 2013; 493:433-6.
46. Sultana N, et al. In Vitro Synthesis of Modified RNA for Cardiac Gene Therapy. Methods Mol Biol. 2021; 2158:281-294.
47. Sultana N, et al. Optimizing Cardiac Delivery of Modified mRNA. Mol Ther. 2017; 25:1306-1315.
48. Tao Z, et al. Dexamethasone inhibits regeneration and causes ventricular aneurysm in the neonatal porcine heart after myocardial infarction. J Mol Cell Cardiol. 2020; 144:15-23.
49. Velayutham N, et al. Cardiomyocyte cell cycling, maturation, and growth by multinucleation in postnatal swine. J Mol Cell Cardiol. 2020; 146:95-108.
50. Wroblewska L, et al. Mammalian synthetic circuits with RNA binding proteins for RNA-only delivery. Nat Biotechnol. 2015; 33:839-41.
51. Xiong Q, et al. Functional consequences of human induced pluripotent stem cell therapy: myocardial ATP turnover rate in the in vivo swine heart with postinfarction remodeling. Circulation. 2013; 127:997-1008.
52. Yan A T, et al. Characterization of the peri-infarct zone by contrast-enhanced cardiac magnetic resonance imaging is a powerful predictor of post-myocardial infarction mortality. Circulation. 2006; 114:32-9.
53. Ye L, et al. Early Regenerative Capacity in the Porcine Heart. Circulation. 2018; 138:2798-2808.
54. Zangi L, et al. Modified mRNA directs the fate of heart progenitor cells and induces vascular regeneration after myocardial infarction. Nat Biotechnol. 2013; 31:898-907.
55. Zangi L, et al. Modified mRNA directs the fate of heart progenitor cells and induces vascular regeneration after myocardial infarction. Nat Biotechnol. 2013; 31:898-907.
56. Zhang E, et al. Identifying the key regulators that promote cell-cycle activity in the hearts of early neonatal pigs after myocardial injury. PLoS One. 2020; 15:e0232963.
57. Zhang L, et al. Derivation and high engraftment of patient-specific cardiomyocyte sheet using induced pluripotent stem cells generated from adult cardiac fibroblast. Circ Heart Fail. 2015; 8:156-66.
58. Zhao M, et al. Apical Resection Prolongs the Cell Cycle Activity and Promotes Myocardial Regeneration After Left Ventricular Injury in Neonatal Pig. Circulation. 2020; 142:913-916.
59. Zhao M, et al. Cyclin D2 Overexpression Enhances the Efficacy of Human Induced Pluripotent Stem Cell-Derived Cardiomyocytes for Myocardial Repair in a Swine Model of Myocardial Infarction. Circulation. 2021; 144:210-228.
60. Zhong W, et al. Hypertrophic growth in cardiac myocytes is mediated by Myc through a Cyclin D2-dependent pathway. Embo j. 2006; 25:3869-79.
61. Zhu W, et al. CCND2 Overexpression Enhances the Regenerative Potency of Human Induced Pluripotent Stem Cell-Derived Cardiomyocytes: Remuscularization of Injured Ventricle. Circ Res. 2018; 122:88-96.
62. Zhu W, et al. Regenerative Potential of Neonatal Porcine Hearts. Circulation. 2018; 138:2809-2816.
63. Zhu W, et al. Targeted expression of cyclin D2 ameliorates late stage anthracycline cardiotoxicity. Cardiovasc Res. 2019; 115:960-965.

What is claimed is:

1. A synthetic modified mRNA (modRNA) system for delivery of at least one cell cycle regulator gene to cardiac cells, wherein the modRNA system comprises a composition comprising at least a first modRNA and a second modRNA;
wherein the first modRNA comprises:
(a) an mRNA sequence complementary to SEQ ID NO. 3;
(b) a recognition sequence for the microRNA miR-1; and
(c) a recognition sequence for the microRNA miR-208; and
wherein the second modRNA comprises:
(d) a kink-turn motif; and
(e) an mRNA sequence complementary to SEQ ID NO. 1.

2. The modRNA system of claim 1, wherein the recognition sequence for miR-1 and the recognition sequence for miR-208 are located 3′ to the mRNA sequence complementary to SEQ ID NO. 3.

3. The modRNA system of claim 1, wherein the kink-turn motif is located 5′ to the mRNA sequence complementary to SEQ ID NO. 1.

4. The modRNA system of claim 1, wherein both the first modRNA and the second modRNA comprise N1-methylpseudouridine residues in place of one or more uridine residues.

5. The modRNA system of claim 1, wherein both the first modRNA and the second modRNA comprise N1-methylpseudouridine residues in place of all uridine residues.

6. The modRNA system of claim 1, wherein the at least one cell cycle regulator gene comprises CCND2.

7. The modRNA system of claim 1, wherein the cardiac cells comprise cardiac fibroblasts, cardiomyocytes, endothelial cells, other cardiac cells, or any combination thereof.

8. The modRNA system of claim 1, wherein the first modRNA and the second modRNA are nonimmunogenic.

9. The modRNA system of claim 1, wherein the modRNA system is inactive in non-cardiac cells.

10. The modRNA system of claim 1, wherein the composition comprises 1.5 μg of the first modRNA and 3 μg of the second modRNA.

11. A method for improving at least one measure of cardiac health in a subject, the method comprising administering the modRNA system of claim 1 to the subject.

12. The method of claim 11, wherein the method is performed after myocardial infarction (MI).

13. The method of claim 11, wherein the modRNA system is administered via intramyocardial injection.

14. The method of claim 11, wherein neither the first modRNA nor the second modRNA is genomically integrated into the subject.

15. The method of claim 11, wherein the method induces transient overexpression of CCND2 in at least one cardiac cell type.

16. The method of claim 15, wherein the at least one cardiac cell type comprises cardiac fibroblasts, cardiomyocytes, endothelial cells, other cardiac cells, or any combination thereof.

17. The method of claim 11, wherein the transient overexpression peaks about 2 days after performing the method and declines to substantially background levels about 14 days after performing the method.

18. The method of claim 11, wherein the at least one measure of cardiac health comprises increased expression of cell cycle markers, enhancement of cardiac function, promotion of myocardial repair, or any combination thereof.

19. The method of claim 11, wherein the subject is a mammal.

20. The method of claim 11, wherein the mammal is a human, mouse, or pig.
